(19) United States
(12) Patent Application Publication
(10) Pub. No.: US 2015/0250892 A1
(43) Pub. Date: Sep. 10, 2015

(54) BIODEGRADABLE POLYMERIC BUFFERS

(71) Applicant: Northeastern University, Boston, MA (US)

(72) Inventors: Mansoor M. AMIJI, Attleboro, MA (US); Arun K. IYER, Boston, MA (US)

(21) Appl. No.: 14/588,808

(22) Filed: Jan. 2, 2015

Related U.S. Application Data
(63) Continuation of application No. PCT/US2013/049114, filed on Jul. 2, 2013.
(60) Provisional application No. 61/667,170, filed on Jul. 2, 2012.

(51) Int. Cl. A61K 47/36; A61K 31/713; A61K 31/7048; C08B 37/08; A61K 9/48

(52) U.S. Cl. CPC A61K 47/36; C08B 37/0072; A61K 9/4816; A61K 31/7048; A61K 31/713

(57) ABSTRACT

Biodegradable pH altering polymers are disclosed. In accordance with certain aspects, the biodegradable pH altering polymers may be used to alter the pH of a microenvironment. In accordance with other aspects, the biodegradable pH altering polymers are utilized for targeted drug and gene delivery and their spontaneous release in intracellular sites of interest.

### CROSS-REFERENCE TO RELATED APPLICATIONS

This application is a continuation of International Application No. PCT/US2013/049114, filed on Jul. 2, 2013, and published on Jan. 9, 2014 as WO 2014/008283, which claims the benefit of U.S. Provisional Patent No. 61/667,170, filed Jul. 2, 2012, the entire contents of each of which are hereby incorporated by reference herein.

### STATEMENT REGARDING FEDERALLY SPONSORED RESEARCH/DEVELOPMENT

The present invention was made with United States government support under Grant Nos. U01-CA15142 and U54-CA15188 awarded by the National Cancer Institute. The United States government has rights in this invention.

### FIELD

The present application relates to biodegradable polymeric buffers and, more particularly, to the use thereof for effecting intracellular pH alteration or buffering action.

### BACKGROUND

Delivery of nucleic acid therapies to specific disease tissue and cells in the body is challenging due to large molecular weight, negative charge, and relatively poor stability especially in biological fluids that are rich in degrading enzymes (such as DNAse and RNAse). Many technologies for nucleic acid delivery utilize cationic lipids and polymers that form electrostatic complexes (such as lipoplexes and polyplexes) with negatively-charged nucleic acid constructs. These cationic systems can be inefficient for gene therapy (with plasmid DNA) or RNA interference therapy (with siRNA) due to lack of intracellular release and stability. In addition, cationic lipids and polymers can be toxic to cells and tissues.

It would be beneficial to be able to use biodegradable polymers for effecting intracellular pH alteration or buffering action to trigger drug/oligonucleotide release.

### SUMMARY

The present application is directed to biodegradable pH altering polymers. In accordance with certain aspects, the biodegradable pH altering polymers may be used to alter the pH of a microenvironment. In accordance with other aspects, the biodegradable pH altering polymers are utilized for targeted drug and gene delivery and their spontaneous release in intracellular sites of interest. In accordance with certain aspects, the biodegradable polymer with pH altering functions is capable of protecting an encapsulated payload during delivery and also facilitating internalization, and targeted release of the drug/payload in specific organelles or regions within the cells. Targeted release may be through a variety of mechanisms. For example, targeted release may be by degradation of the polymer matrix due to micro-environmental pH changes or due to the inherent pH change caused by the polymeric nanosystem, modulated by a change in microenvironmental pH shift.

The pH altering effect may be achieved by controlling the density of pH altering functional groups on the biodegradable/biocompatible polymers thereby altering the pKa of the modified polymers. The pH altering groups such as amine derivatives may be conjugated to biodegradable polymers. Furthermore, fatty amines or hydrophobic molecules containing multiple amino functionalities may be conjugated to the hydrophilic backbone to facilitate self-assembly, to form stable nanoassemblies in solution. Also, cationic polyamine derivatives conjugated onto the biocompatible polymers may be tailored to stably complex DNA/SiRNA and/or drugs ensuring their encapsulation into the polymeric nanosystems without causing apparent toxicity. One of the advantages of such a system relates to the ability to control the composition of the polymer functionality to have the right balance of charge for encapsulating DNA/SiRNA and at the same time maintaining a net neutral or negative charge on the overall polymeric system, thereby rendering no or minimal toxicity to the non-target organs and tissue on in vivo administration.

For this purpose, varying molecular weights of the biodegradable/biocompatible polymers, such as 10, 20, 40 kDa hyaluronic acid (HA) or dextrans with varying chain lengths, may be used to optimize the nanoassemblies. In accordance with certain aspects, the molecular weight of the HA derivatives may be in the range of about 10-100 kDa, more particularly about 10-40 kDa. In accordance with particularly useful aspects, the charge on the polymer may fall in the range from about −40 to +10 mV. Furthermore, the hydrophilic/hydrophobic balance on the polymer, degree of functionalization and the number of amino groups may be controlled by altering the reaction conditions as well as by using various fatty amine derivatives with varying carbon chain lengths. Examples of suitable derivatives include, but are not limited to, butyl amine (C=4), hexyl amine (C=6), octyl amine (C=8) and stearyl amine (C=18). Derivatives with multiple nitrogen groups such as 1,3 diamine propane (C=10, N=4), 1,4 diamino butane (C=4, N=2), 1,6 diamino hexane (C=6, N=2) 1,2 aminoethyl piperazine (C=6, N=3) and spermine (C=10, N=4) may be used to arrive at the right combination of charge for SiRNA/drug encapsulation and stable self-assembly for formation of nanoparticles. In some cases, protected fatty acids such as BOC-1,4) diamino butane or BOC-1,3 diamino propane can be used to conjugate with a biodegradable polymer (e.g., hyaluronic acid) followed by deprotection to yield the free primary amine containing derivatives. These derivatives may further be modified via the free primary amine groups to form derivatives with variable carbon chain lengths containing nitrogen atoms in the backbone. In other cases, azide-alkyne click chemical ligations may be used to create HA based functional macrostructure with precise control on the density of lipid tails and/or charge density of polyamines.

The present application also describes the pH altering functions of the nanosystems in vitro on incubation with cells and their effect on the release of the SiRNA/drug in the target sites within the cells.

In one aspect, the present application provides a method of modifying the pH of a microenvironment in need of pH modification by introducing into the microenvironment a polymeric buffer, wherein the polymeric buffer comprises a biodegradable pH altering polymer or a biodegradable polymer conjugated with pH altering groups. In accordance with certain aspects, the pH altering groups may include at least one nitrogen-containing group and in certain cases, at least one amine group. The polymeric buffer may include primary, secondary or tertiary nitrogen containing molecules. The polymeric buffer may have a pKa of at least 3.0, more particularly from about 4.0-6.0, and, still more particularly, from about 4.5-5.0.

In another aspect of the present application, the pH altering groups on the polymeric buffer may be derivatives of butyl amine, hexyl amine, octyl amine, stearyl amine, 1,3 diamine propane, 1,4 diamino butane, 1,6 diamino hexane, 1,2 aminoethyl piperazine or spermine.

The biodegradable polymer in accordance with particular embodiments may be water soluble. Specific examples include, but are not limited to, hyaluronic acid (HA) or dextran. The % modification of the biodegradable polymer may be at least 5%, more particularly from about 10% to 80%, and in certain aspects, from about 10% to 40%. Specific examples of polymeric buffers include, but are not limited to, HA-butylamine, HA-hexylamine, HA-octylamine, HA-1-amino decane, HA-stearylamine, HA-oleyl amine, HA-1,6 diaminohexane, HA-1,8 diaminooctane, HA-choline, HA-polyethyleneimine and HA-spermine.

In accordance with another embodiment, pharmaceutical compositions are provided comprising a pharmaceutically-acceptable carrier or diluent and at least one polymeric buffer, wherein the polymeric buffer comprises a biodegradable pH altering polymer or a biodegradable polymer conjugated with pH altering groups. The pharmaceutical composition may include a therapeutic agent. In accordance with certain aspects, the therapeutic agent may be a chemotherapeutic agent. In particular embodiments, the chemotherapeutic agent is doxorubicin, paclitaxel, or tamoxifen. In some embodiments, the therapeutic agent is a functional nucleic acid or a functional nucleic acid construct. In particular embodiments, the nucleic acid is an siRNA molecule, an aptamer, or a ribozyme.

The present application is also directed to a method of treating a subject having a tumor. In accordance with one aspect, the method comprising administering to the subject a composition containing nanoparticles in an amount sufficient to reduce tumor size or number of tumor cells in the tumor, wherein the nanoparticle includes a therapeutic agent and a hydrogel shell surrounding the therapeutic agent. The hydrogel shell includes a biodegradable pH altering polymer or a biodegradable polymer conjugated with pH altering groups.

In accordance with another aspect, the present application is directed to a method of inhibiting expression of a target polypeptide in a subject. In accordance with one embodiment, a composition containing nanoparticles in an amount sufficient to inhibit expression of the target polypeptide is administered to a subject in need of treatment. The nanoparticle includes nucleic acid and a hydrogel shell surrounding the nucleic acid, wherein the hydrogel shell comprises a biodegradable pH altering polymer or a biodegradable polymer conjugated with pH altering groups.

### BRIEF DESCRIPTION OF THE DRAWINGS

FIG. 1 illustrates one scheme for producing hyaluronic acid derivatives in accordance with one embodiment;

FIG. 2 illustrates another scheme for producing hyaluronic acid derivatives in accordance with another embodiment;

FIG. 3 illustrates a scheme for producing hyaluronic acid derivatives in accordance with yet another embodiment;

FIG. 4 illustrates another scheme for producing hyaluronic acid derivatives in accordance with another embodiment;

FIG. 5 illustrates another scheme for producing hyaluronic acid derivatives using DCC coupling followed by deprotection in accordance with still another embodiment;

FIG. 6 illustrates a proposed structure of PEI-modified HA following self-assembly with siRNA;

FIG. 7 shows the ¹H-NMR Spectra of the native HA polymer, native PEI, HA and PEI mixture and a purified HA-PEI conjugate in accordance with one embodiment;

FIG. 8 illustrates a scheme for “clickable” hyaluronic acid based functional macrostructures in accordance with one embodiment;

FIG. 9 shows the ¹H-NMR Spectra of various HA based functional polymers in accordance with certain embodiments;

FIG. 10 shows the ¹H-NMR Spectra comparison of Hyaluronic acid (20 kDa) (A) with Hyaluronic acid (20 kDa)-Oleyl amine (C₁₈N₁) (B);

FIG. 11 shows the ¹H-NMR Spectra comparison of Hyaluronic acid (20 kDa) (A) with Hyaluronic acid (20 kDa)-1,8 diamino octane (C₈N₂) (B);

FIG. 12 shows the ¹H-NMR Spectra comparison of Hyaluronic acid (20 kDa) (A) with Hyaluronic acid (20 kDa)-Spermine (C₁₀N₄);

FIG. 13 shows acid-base titration curves of unmodified hyaluronic acid (10 kDa);

FIG. 14 shows acid-base titration curves of unmodified hyaluronic acid (20 kDa);

FIG. 15 shows acid-base titration curves of hyaluronic acid polymer modified with oleyl amine (HA-OA);

FIG. 16 shows acid-base titration curves of hyaluronic acid polymer modified with 1-aminodecane;

FIG. 17 shows acid-base titration curves of hyaluronic acid polymer modified with 1,8 diamino octane (HA-ODA);

FIG. 18 shows acid-base titration curves of hyaluronic acid polymer modified with spermine;

FIG. 19 provides TEM images of Paclitaxel and Docetaxel nanoparticles;

FIG. 20 shows fluorescence microscopy of free doxorubicin (DOX), DOX loaded unmodified hyaluronic acid (HA-DOX, PKa˜3) polymer and DOX loaded HA polymer modified with 1,8 diamino octane (HA-ODA-DOX, PKa˜5) at a magnification of 40×;

FIGS. 21A-C provide a putative representation of the formation of an siRNA-loaded HA-nanosystem;

FIG. 22 shows electrophoretic retardation analysis of an siRNA binding by HA-PEI derivatives at different mass ratios (90:1, 54:1, 45:1). The release of intact siRNA by polyacrylic acid was shown in each case;

FIG. 23 provides confocal microscopy images of MDAMB-468 cells after treatment with HA PEI/Cy3siRNA at 50 nM for 12 h. The internalization of siRNA could be clearly seen in the cells (red signal);

FIG. 24 provides confocal microscopy images of cells incubated with HA-PEI/Cy3-labeled siRNA in the presence and absence of excess free HA;

FIGS. 25A-B show cellular uptake of HA-choline/cy3 siRNA in MDA-MB 468 cells. For competitive inhibition study, the cells were incubated with HA-choline in the presence and absence of excess free HA;

FIG. 26 is a graph of PLK1 gene expression for various treatment groups showing the effects of chloroquine;

FIGS. 27A-B are graphs of PLK1 gene expression for various treatment groups evaluating the ability of complexes to deliver a functional siRNA;

FIGS. 28A-B are graphs of PLK1 gene expression for HA-PEI complexes under various conditions;

FIG. 29 is a graph showing PLK1 siRNA knockdown in A549/A549^{DDP} NSCL cancer cells;

FIG. 30 depicts live animal imaging and tumor targeting of HA based nanosystems;

FIG. 31 is a chart of tissue distribution of surviving siRNA in A549^{DDP} tumor bearing mice;

FIG. 32 is a chart showing the in vivo gene silencing at varying time points;

FIGS. 33A-C provide graphs showing the efficacy of combination siRNA and Cisplatin loaded in HA Nanosystems; and

FIG. 34 presents in vivo safety evaluation results of the HA Nanosystems.

### DETAILED DESCRIPTION

### Definitions

All publications, patent applications, patents, and other references mentioned herein, including GenBank database sequences, are incorporated by reference in their entirety. Unless otherwise defined, all technical and scientific terms used herein have the same meaning as commonly understood by one of ordinary skill in the art to which this invention belongs. In case of conflict, the present specification, including definitions, will control. In addition, the materials, methods, and examples are illustrative only and not intended to be limiting. Although methods and materials similar or equivalent to those described herein can be used in the practice or testing of the present invention, suitable methods and materials are described below.

The following are definitions of terms used in the present specification. The initial definition provided for a group or term herein applies to that group or term throughout the present specification individually or as part of another group, unless otherwise indicated.

As used herein, “about” means a numeric value having a range of ±10% around the cited value.

As used herein, a “subject” is a mammal, e.g., a human, mouse, rat, guinea pig, dog, cat, horse, cow, pig, or non-human primate, such as a monkey, chimpanzee, baboon or rhesus.

As used herein, the term “biodegradable” refers to a substance that is decomposed (e.g., chemically or enzymatically) or broken down in component molecules by natural biological processes (e.g., in vertebrate animals such as humans).

As used herein, the term “biocompatible” refers to a substance that has no unintended toxic or injurious effects on biological functions in a target organism.

As used herein, the term “nanoparticle” refers to a particle having a diameter in the range of about 50 nm to about 1000 nm. Nanoparticles include particles capable of containing a therapeutic or imaging agent that can be released within a subject.

As used herein, the terms “conjugated,” “derivatized,” and “linked” are used interchangeably, and mean that two components are physically linked by, for example, covalent chemical bonds or physical forces such van der Waals or hydrophobic interactions. Two components can also be conjugated indirectly, e.g., through a linker, such as a chain of covalently linked atoms.

As used herein, “treat,” “treating” or “treatment” refers to administering a therapy in an amount, manner (e.g., schedule of administration), and/or mode (e.g., route of administration), effective to improve a disorder (e.g., a disorder described herein) or a symptom thereof, or to prevent or slow the progression of a disorder (e.g., a disorder described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

An “effective amount,” when used in connection with a composition described herein, is an amount effective for treating a disorder or a symptom thereof.

The term “polymer,” as used herein, refers to a molecule composed of repeated subunits. Such molecules include, but are not limited to, polypeptides, polynucleotides, polysaccharides or polyalkylene glycols. Polymers can also be biodegradable and/or biocompatible.

The terms “polypeptide,” “peptide,” and “protein” are used interchangeably herein and refer to a polymer of amino acid residues. The terms apply to naturally occurring amino acid polymers as well as amino acid polymers in which one or more amino acid residues are non-natural amino acids. Additionally, such polypeptides, peptides, and proteins include amino acid chains of any length, including full length proteins, wherein the amino acid residues are linked by covalent peptide bonds.

The term “drug” or “therapeutic agent,” as used herein, refers to any substance used in the prevention, diagnosis, alleviation, treatment, or cure of a disease or condition.

The term “targeting agent” refers to a ligand or molecule capable of specifically or selectively (i.e., non-randomly) binding or hybridizing to, or otherwise interacting with, a desired target molecule. Examples of targeting agents include, but are not limited to, nucleic acid molecules (e.g., RNA and DNA, including ligand-binding RNA molecules such as aptamers, antisense, or ribozymes), polypeptides (e.g., antigen binding proteins, receptor ligands, signal peptides, and hydrophobic membrane spanning domains), antibodies (and portions thereof), organic molecules (e.g., biotin, carbohydrates, and glycoproteins), and inorganic molecules (e.g., vitamins). A nanoparticle described herein can have affixed thereto one or more of a variety of such targeting agents.

As used herein, “self assembly,” “self-assembled,” or “self-assembling” means that components assemble into a nanoparticle without the application of a physical force, such as sonication, high pressure, membrane intrusion, or centrifugation.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of a compound of the present invention may be formed, for example, by reacting a compound I with an amount of acid or base, such as an equivalent amount, in a medium such as one in which the salt precipitates or in an aqueous medium followed by lyophilization.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% pure (“substantially pure” compound I), which is then used or formulated as described herein. Such “substantially pure” compounds of the present invention are also contemplated herein as part of the present invention.

Throughout the specifications, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito: 1999, the entire contents of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

The amount of a compound according to the present invention, also referred to here as the active ingredient, which is required to achieve a therapeutic effect may vary on case-by-case basis, vary with the particular compound, the route of administration, the age and condition of the recipient, and the particular disorder or disease being treated. A method of treatment may also include administering the active ingredient on a regimen of between one and four intakes per day. In these methods of treatment the compounds according to the invention are preferably formulated prior to admission. As described herein below, suitable pharmaceutical formulations are prepared by known procedures using well known and readily available ingredients.

In certain instances, neutral or negatively-charged water-soluble biodegradable and/or biocompatible polymers are used. These include, without limitation, dextran, polysaccharides, polypeptides, polynucleotides, acrylate gels, polyanhydride, poly(lactide-co-glycolide), polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homoploymers, polyoxanones and polyoxalates, copolymer of poly(bis(p-carboxyphenoxy) propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), polydihydropyrans, polyphosphazenes, poly(ortho ester), poly(cyano acrylates), poly-depsipeptides, lactide-depsipeptides polymers, depsipeptide-co-polymers, polyvinylpyrrolidone, polyvinylalcohol, poly casein, keratin, myosin, and fibrin, or polyurethanes, and the like. Other neutral or negatively-charged water-soluble polymers that can be used include naturally derived polymers, such as acacia, gelatin, dextrans, albumins, alginates/starch, and the like; or synthetic polymers, whether hydrophilic or hydrophobic. Examples of other polymers useful for drug delivery include low MW oligomers of styrene-maleic acid and their co-polymers that are water soluble but not biodegradable. The materials can be synthesized, isolated, and are commercially available.

Other features and advantages of the invention will be apparent from the following detailed description, and from the claims.

### Methods of Preparation:

### Pharmaceutical Compositions

This invention also provides a pharmaceutical composition comprising at least one of the polymeric buffers as described herein or a pharmaceutically-acceptable salt thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra.)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples are embedding compositions, which can be used include polymeric substances and waxes. The active ingredient can also be in micro-encapsulated form with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams or spray formulations containing such carriers as are known in the art to be apbutriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the buter medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another agent for treating the same disorder), or they may achieve different effects (e.g., control of any adverse effects).

The compounds of the invention may be administered intravenously, intramuscularly, intraperitoneally, subcutaneously, topically, orally, or by other acceptable means. The compounds may be used to treat conditions in mammals (i.e., humans, livestock, and domestic animals), birds, lizards, and any other organism, which can tolerate the compounds. The compositions can be introduced to target areas in need of pH alteration for a given microenvironment.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diseases and Disorders

The nanoparticles described herein can be used to treat (e.g., mediate the translocation of drugs into) diseased cells and tissues. In this regard, various diseases are amenable to treatment using the nanoparticles and methods described herein. An exemplary, nonlimiting list of diseases that can be treated with the subject nanoparticles includes breast cancer; prostate cancer; lung cancer; lymphomas; skin cancer; pancreatic cancer; colon cancer; melanoma; ovarian cancer; brain cancer; head and neck cancer; liver cancer; bladder cancer; non-small lung cancer; cervical carcinoma; leukemia; non-Hodgkins lymphoma, multiple sclerosis, neuroblastoma and glioblastoma; T and B cell mediated autoimmune diseases; inflammatory diseases; infections; infectious diseases; hyperproliferative diseases; AIDS; degenerative conditions; cardiovascular diseases (including coronary restenosis); diabetes; transplant rejection; and the like. In some cases, the treated cancer cells are metastatic.

In particular instances, a nanoparticle described herein can be used to reverse multi-drug resistance (MDR). For examples, downregulation of MDR transporter and anti-apoptotic genes such as Bcl-2, survivin, mdr-1, or mrp-1 by siRNA-containing nanoparticles can be used.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

### Synthesis of Hyaluronic Acid Derivatives

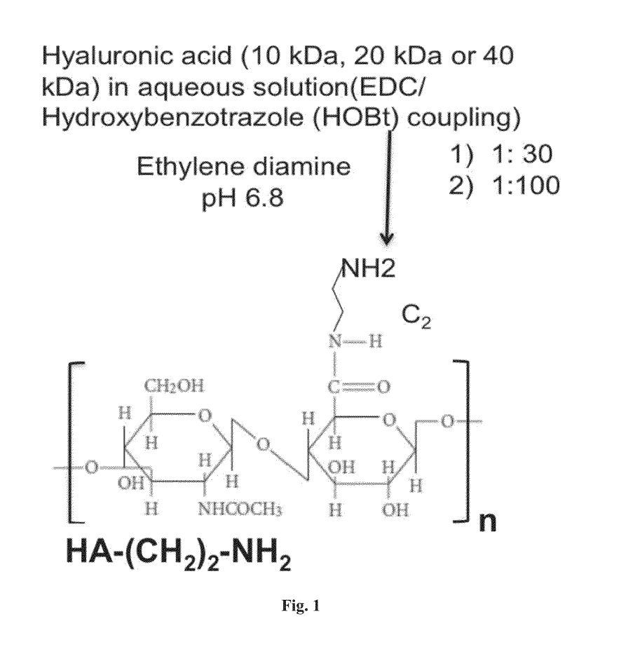

### Scheme 1 (See FIG. 1):

Sodium hyaluronate (100 mg, 0.25 mmol, mw 10 kDa/20 kDa/40 kDa) was dissolved in water at a concentration of 3 mg/ml. To this solution was added a 30 fold excess of an amine or hydrazide (pKa 3-8; 7.5 mmol) e.g., ethylenediamine, adipic hydrazide. The pH of the reaction mixture was adjusted to 6.8 with 0.1 M NaOH/0.1M HCl.

1ethyl-3-[3-(dimethylamino)propyl]carbodiimide (EDC) (192 mg, 1 mmol) and 1-hydroxybenzotriazole (HOBt) (135 mg, 1 mmol) was dissolved in DMSO/water (1:1). After mixing, the pH of the reaction was maintained at 6.8 by the addition of 0.1 M NaOH and the reaction was allowed to proceed overnight. The pH was subsequently adjusted to 7.0 with 0.1 M NaOH and the derivatized hyaluronic acid was dialyzed exhaustively, to yield the purified product. The modified HA was precipitated by addition of 3 vol equivalents of ethanol. The precipitate was redissolved in water at a concentration of 5 mg/ml and the purified product was freeze-dried and kept at 4 degree C. The yield of the product was typically 80%.

### Scheme 2:

To an aqueous solution of sodium hyaluronate (10 kDa/20 kDa/40 kDa, 3 mg/ml) was added a 30 fold molar excess of an amine (7.5 mmol), e.g., 1-4 diamino butane or 1-6 diamino hexane. The pH of the reaction mixture was adjusted to 7.5 with 0.1 M NaOH/0.1 M HCl. EDC (192 mg, 1 mmol) and N-hydroxysulfosuccinimide (Sulfo-NHS) (217 mg, 1 mmol) were dissolved in water (1 ml). After mixing, the pH of the reaction was maintained at 7.5 by the addition of 0.1 M NaOH and the reaction was allowed to proceed overnight. The HA derivatives were purified and stored at 4 degree C.

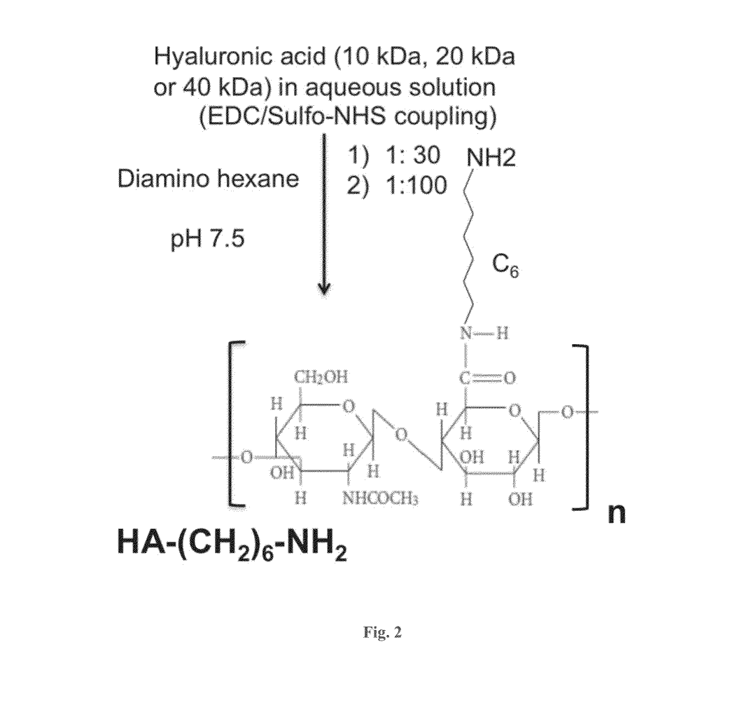

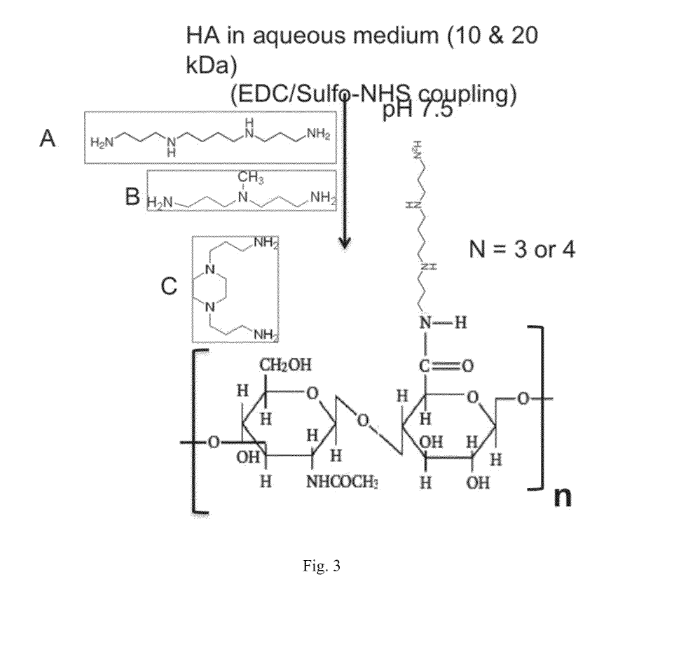

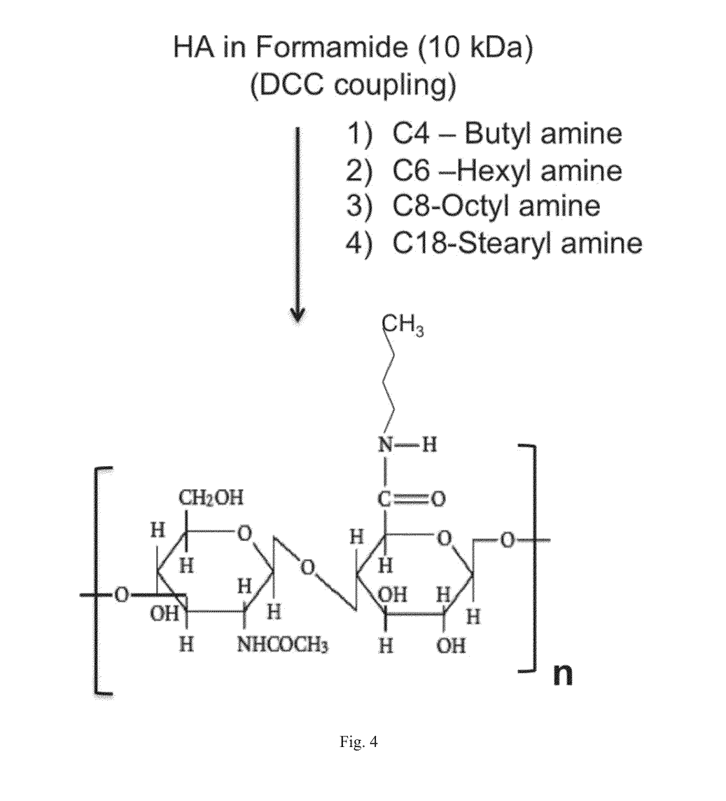

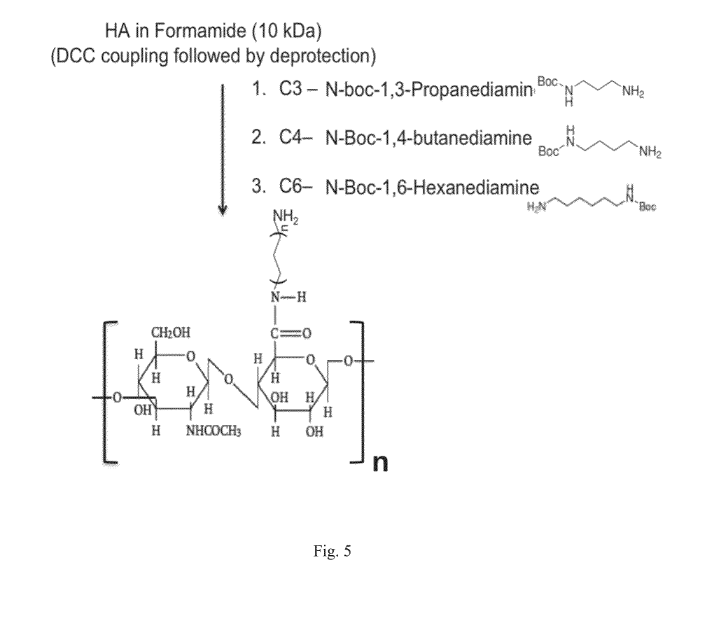

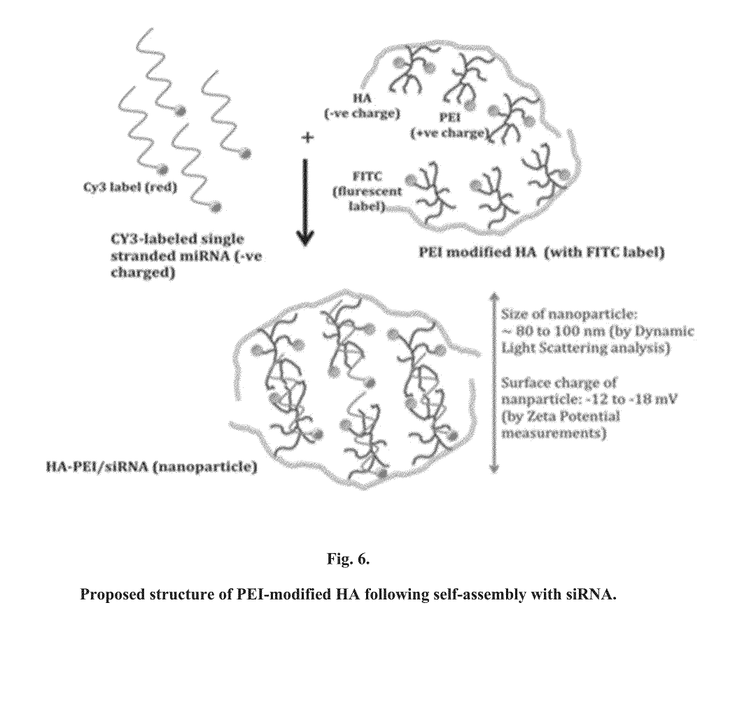

Specific examples are provided in Scheme 2: A (see FIG. 2); Scheme 2: B (see FIG. 3); Scheme 3: A (see FIG. 4); and Scheme 3: B (see FIG. 5).

Scheme 4: Incorporating Polyamines Containing Secondary or Tertiary Amines with Higher Degree of Modifications.

For gene delivery charge interaction plays a key role and by altering the reaction conditions one can improve the percentage modification of spermine on the surface of HA. To address this, the polymer can be reacted with polyamines in THF in the presence of DCC/NHS with the assumption that the modifications can be increased without additional cross linkings and that would further overcome the negative charges on the HA surface and thereby enhance the encapsulation, cell entry and endosome escape to ultimately show improved silencing.

Scheme 4.2: Modifying HA with PEI

With the intention of increasing the encapsulation/endosome release and activity, the HA backbone was modified with a highly cationic polyamine, poly(ethyleneimine) (PEI) under mild reaction conditions without generating any crosslinking PEI has multiple amine groups that seem to efficiently condense with siRNA and form a core within self-assembled particles. On the complexation with siRNA, the zeta potential was inverted from positive for the PEI to negative for the siRNA/HA-PEI, reflecting the core-shell structure of the HA-PEI/siRNA complex with HA backbone exposed in the shell and the PEI grafted chains complexed with RNA molecules in the core.

Hyaluronic acid (HA) polymer was chemically modified with polyethyleneimine (PEI) by using a coupling agent, 1-Ethyl-3-[3-dimethylaminopropyl]carbodiimide hydrochloride (EDC). In brief, sodium hyaluronate (MW 20 kDa, 100 mg, 5 μmole, Lifecore Biomedical, Chaska, Minn.) was dissolved in 5 ml of dry formamide in a glass scintillation vial by warming up the reaction vial up to 50° C. After obtaining a clear solution the reaction mixture was allowed to cool to room temperature and then ˜3.3 mg of the PEI (Polysciences Inc, Warrington, Pa., MW 10 kDa, ˜0.33 μmole) was added to the solution. Then, EDC (10 μmole, Sigma Aldrich, Mo.) was added into the reaction mixture and stirred for 12 hours using a magnetic stirrer. The resulting reaction mixture was added into a large excess of EtOH (200 ml) to precipitate the polymer. The EtOH washing step was repeated thrice to purify the polymer. Subsequently, the HA-PEI precipitate was further dialyzed using cellulose dialysis membranes (MW cut off ˜12-14 kDa) against deionized water for 96 hours. The purified product was then lyophilized and stored (yield: 90 mg, ˜86%, off-white fibrous product). A 3 mg portion of the lyophilized product was dissolved in 600 μl of D₂O and characterized by 400 MHz ¹H-NMR spectroscopy (Varian Inc., CA).

FIG. 5 illustrates a proposed structure of PEI-modified HA following self-assembly with siRNA. By designing the complexes to include the HA molecules in the outer shell, one can explore the targeting properties of HA. Moreover, the negative charges present on the surface of HA can effectively shield the positive charges of the RNA/PEI complex, which leads to a decrease in toxicity that is normally associated with positively charged molecules.

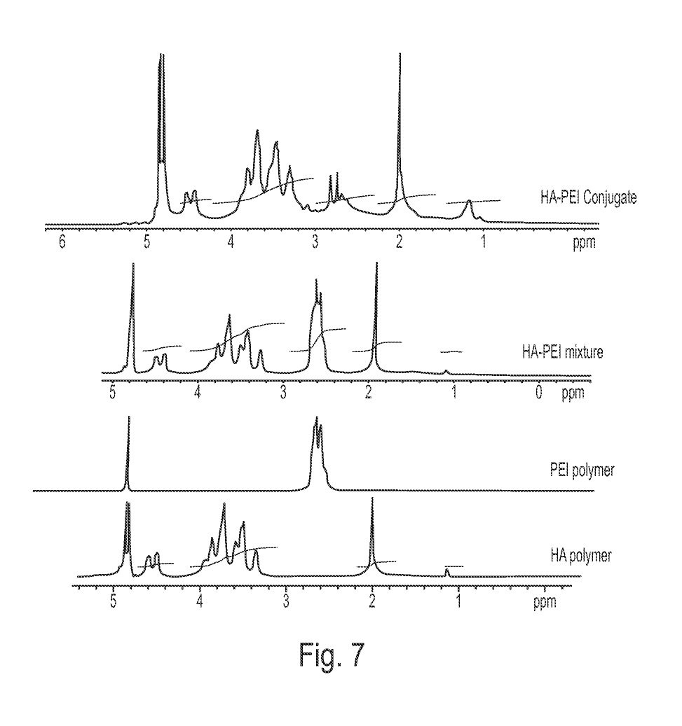

FIG. 7 shows the ¹H-NMR spectra of the native HA polymer, native PEI, HA and PEI mixture and the purified HA-PEI conjugate.

### Scheme 6: ‘Clickable’ HA Based Functional Macrostructures for Stable Self-Assembly and Encapsulation of Diverse Drug Payloads.

Materials:

Sodium hyaluronate (molecular weight 40K, 20K and 10K) was obtained from Lifecore Biomedical. Copper sulfate, bromoethane, bromobutane, bromohexane, bromooctane, bromodecane, bromododecane, bromooctadecane, dimethylformamide (DMF), tetrahydrofuran (THF), and hexane were purchased from Fisher Scientific. N-Ethyl-N′-(3-dimethylamineproyl)-carbodiimide (EDAC) was obtained from Bachem. N-hydroxysulfosccinimide (sulfo-NHS), sodium L-ascorbate, sodium azide, and thiazolyl blue tetrazolium bromide (MTT) were purchased from Sigma Aldrich. 1-Hydroxybenzotriazole (HOBT) was purchased from AK Scientific.

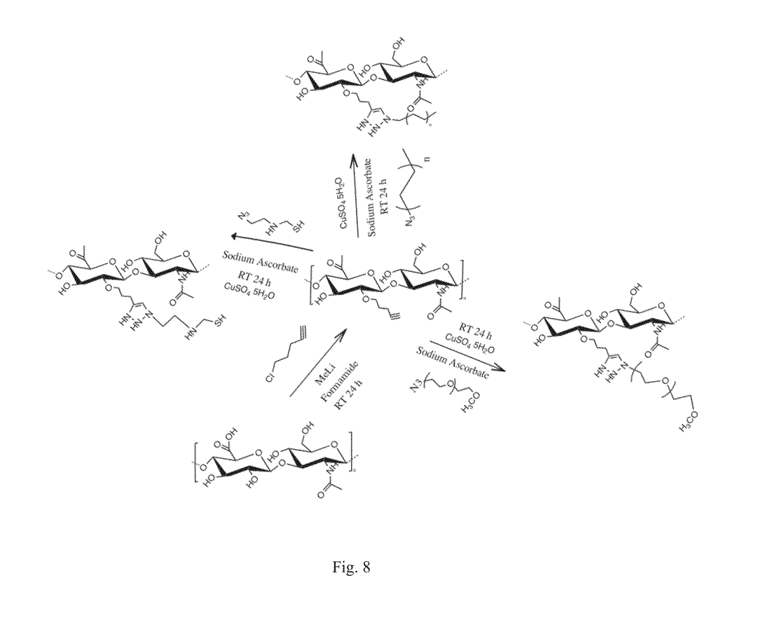

FIG. 8 shows a scheme for “clickable” HA based functional macrostructures.

### Methods:

### Synthesis of “Clickable” HA

For “click” conjugation of hyaluronic acid (HA), an o-pentynyl moiety was attached as shown in Scheme 1. HA (Mw=20 kDa, 1 g, 50 μmol) was dissolved in dry formamide solution (40 mL) at 50° C. after bubbling N₂ into the solution for 15 min. After mixing to form a clear solution, 1.5 M MeLi (18.5 mL, 1.5 eq.) was added. The mixture was cooled in an ice-bath and 5-chloro-1-pentyne (0.6 mL, 0.3 eq.) was added slowly. Stirring was continued for 24 h under nitrogen atmosphere. The product was isolated by precipitating in 350 mL of ethanol, washing two times with 50 mL of ethanol, and purifying by dialysis against demineralized water and freeze drying (1.25 g, white solid) with degree of substitution (DS) ˜10%. Degree of substitution of 10 was determined by means of 1H-NMR.

In accordance with another method to form the alkynyl-HA precursor, propargylamine, an alkynyl moiety, was attached as using EDC/sulfo NHS coupling. Briefly, HA (Mw=20 kDa, 1 g, 6.17 mol) was dissolved in 10 ml water. To it 5 m. of propargylamine was added and stirred. To it 2 molar excess of EDC/sulfo NHS was first dissolved in 5 ml of water and reacted for 30 min to 1 h. Subsequently, EDC/NHS mixture was added (1 ml each time drop-wise addition) to the vial containing HA and propargylamine. The reaction was allowed to proceed overnight followed by purification using TFF system and lyophilization. The degree of substitution was determined by means of ¹H-NMR spectroscopy.

“Click” Synthesis of Lipid-Modified HA

Experiments were carried out with alkynyl HA and alkyl bromides (CₙH₂ₙ₊₁, n=2, 4, 6, 8, 10, 12, 18). In a representative experiment, HA (DS 10%, 250 mg) was dissolved in water (25 ml) and added to a round-bottom flask containing bromoethane (2 g, 18.35 mmol), sodium azide (2.38 g, 36.71 mmol), and copper(II) sulfate pentahydrate (8 mg, 0.032 mmol), sodium ascorbate (19 mg, 0.129 mmol). After stirring the mixture at room temperature for 24 h, product was purified by dialysis against demineralized water and freeze-dried (180 mg, pale green solid).

### “Click” Synthesis of Thiol-Modified HA

Sodium azide (NaN₃, 0.5 g, 3.17 mmol) was added to a solution of 1-bromo-3-chloropropane (0.2 g, 3.17 mmol) in 15 mL of DMF at room temperature. The reaction mixture was allowed to stir for overnight. The reaction mixture was partitioned between ether and water, and the organic layer was washed with water, dried over Na₂SO₄ and concentrated to give 1-azido-3-chloropropane (0.3 g, 92%) as a colorless viscous liquid. Solution of cysteamine (0.19 g, 2.5 mmol) in THF (15 ml) was added to a stirred suspension of 1-azido-3-chloropropane (0.3 g, 2.5 mmol) in THF (15 ml). After stirring under nitrogen for 3 days at room temperature, the solvent was evaporated in vacuo and the yellow solid residue was washed with THF/hexane (1/5). This product was dissolved in 25 ml water and added to a round-bottom flask containing alkynyl HA (DS 10%, 250 mg), copper(II) sulfate pentahydrate (8 mg, 0.032 mmol), sodium ascorbate (19 mg, 0.129 mmol). After stirring the mixture at rt for 24 h, product was purified by dialysis against demineralized water and freeze-dried.

### “Click” Synthesis of PEG-Modified HA

Methoxypolyethylene glycol azide 2000 (250 mg) and alkynyl HA (DS 10%, 250 mg) were dissolved in 25 ml water in a round-bottom flask. Copper(II) sulfate pentahydrate (8 mg, 0.032 mmol) and sodium ascorbate (19 mg, 0.129 mmol) were added and stirred the mixture at rt for 24 h. The product was purified by dialysis against demineralized water and freeze-dried.

### Chemical Synthesis of PEG-Modified HA

HA (MW 20 kDa, 200 mg, 10 μmole) was dissolved in 10 mL of deionized water. HOBt (20 μmol, 3 mg) solubilized in 100 μL of methanol was added drop-wise into the HA solution. EDC (20 μmol, 4 mg) and sulfo-NHS (20 μmol, 4 mg) were mixed together in 5 mL deionized water and allowed to stand for 15 min. The resultant EDC/sulfo-NHS solution was added drop-wise into the HA and HOBt solution for about 1 h. After stirring for 4 h, mPEG-NH₂ solution (2 kDa, 20 mg, 10 μmol) in deionized water (5 mL) was slowly added to the stirred solution. This mixture was stirred for 1 day at room temperature. The resulting solution was then loaded into a dialysis bag (MWCO; 6-8 kDa) and dialyzed against the methanol and DDW mixture (1:1, v/v) for 2 days. After freeze-drying, a yellowish white powder was obtained as a product. The introduction ratio of PEG to HA was measured by ¹H-NMR analysis. HA and HA-PEG were dissolved in D₂O for analysis by ¹H-NMR (400 MHz).

Synthesis of Functional Block with Varying Lipid Tails:

A) HA-Lipid (Hydrophobically Modified HA)

| | | Character- |
|---|---|---|
| Alkynyl HA (mol wt) | Lipid/fatty acid (Mole Ratios) | ization |
| HA(10, 20, 40 kDa) | C₂, C₄, C₆, C₈, C₁₀, C₁₂, C₁₈ | ¹H-NMR |
| | (10, 20, 30 Mole %) | |

5)¹H-NMR Spectroscopy Characterization:

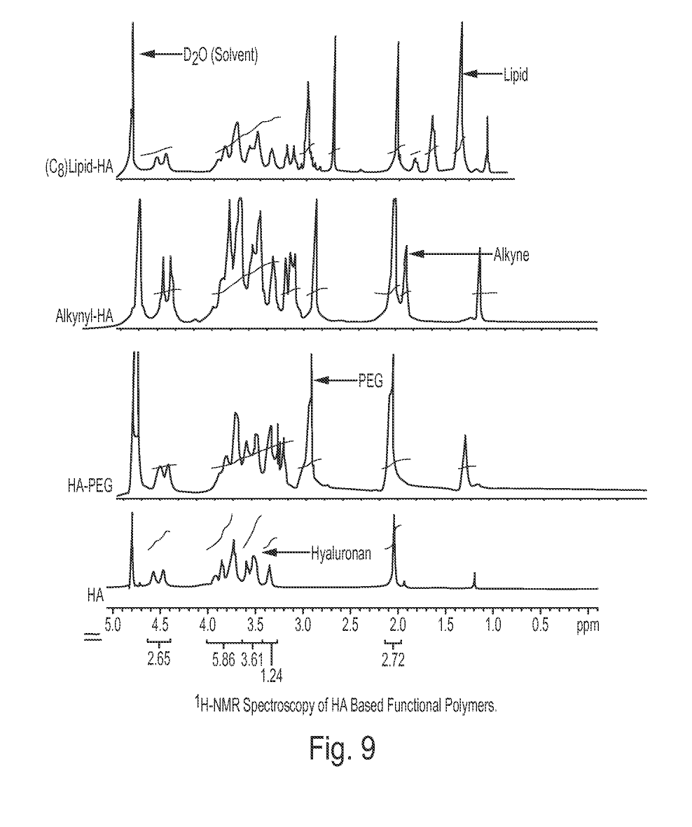

FIG. 9 provides ¹H-NMR Spectroscopy of HA Based Functional Polymers. The figure shows the 1H-NMR spectra of HA and the synthesized HA based polymers after purification. The additional peaks with corresponding chemical shifts for the lipid and PEG modification of HA are clearly seen.

¹H-Nuclear Magnetic Resonance (′H-NMR) spectra of some of the derivatives:

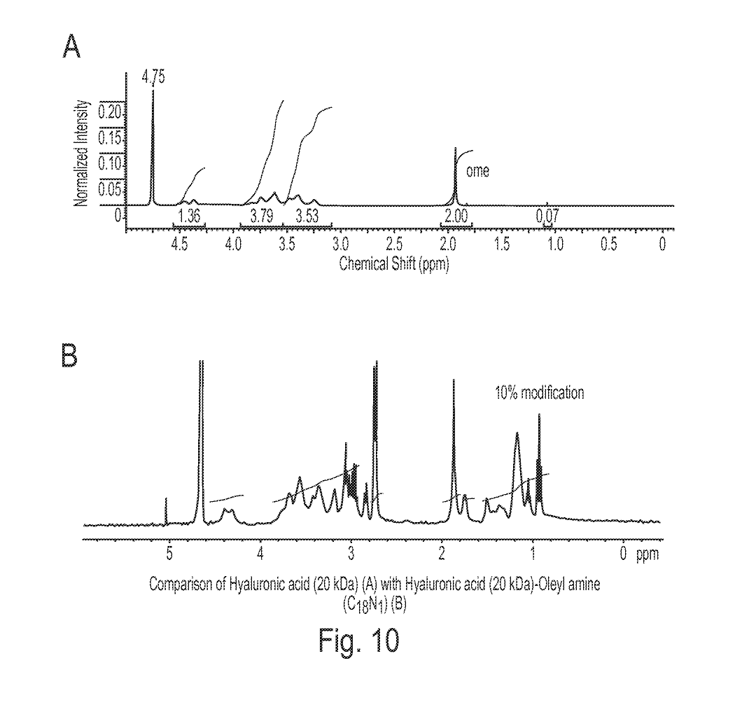

FIG. 10 provides a comparison of Hyaluronic acid (20 kDa) (A) with Hyaluronic acid (20 kDa)-Oleyl amine (C₁₈N₁) (B).

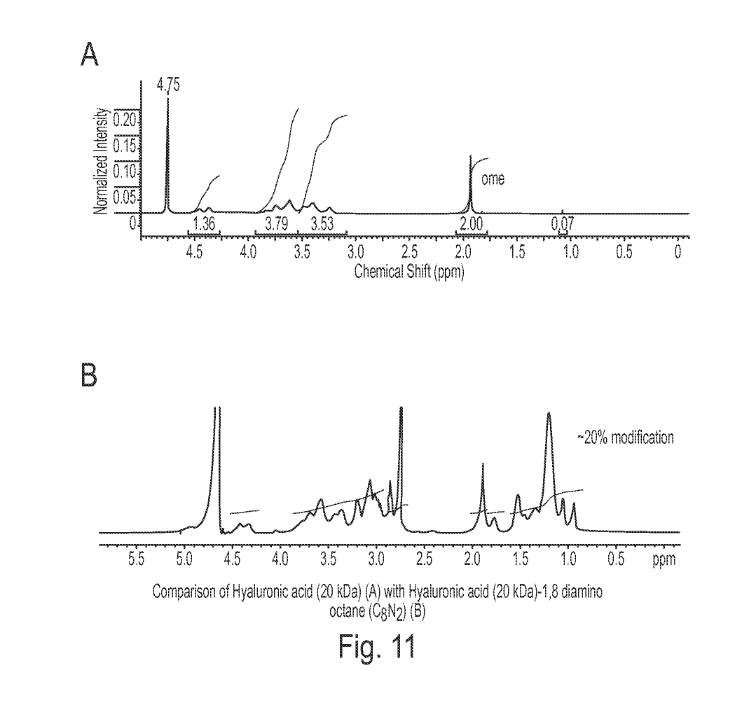

FIG. 11 provides a comparison of Hyaluronic acid (20 kDa) (A) with Hyaluronic acid (20 kDa)-1,8 diamino octane (C₈N₂) (B).

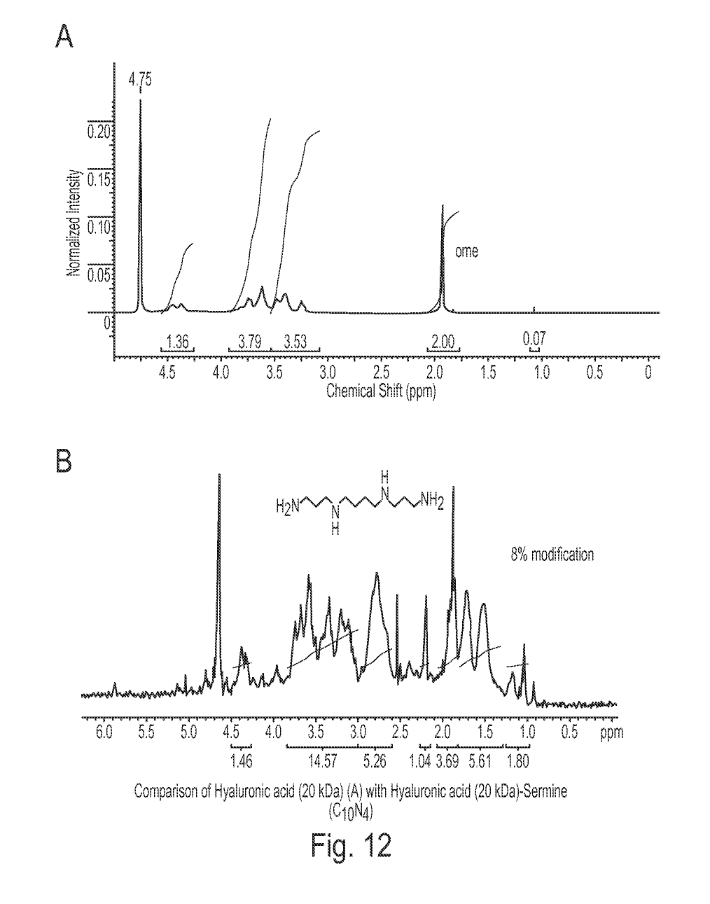

FIG. 12 provides a comparison of Hyaluronic acid (20 kDa) (A) with Hyaluronic acid (20 kDa)-Spermine (C₁₀N₄).

6) Buffering Effects of Representative Hyaluronic Acid (HA) Derivatives

Acid-base titration curves of hyaluronic acid (HA) and hyaluronic acid derivatives.

Method: 50 mg of hyaluronic acid or the purified polymers was dissolved in 5 ml of deionized water and titrated with 100 μl 0.1 M NaOH or 0.1 N HCl

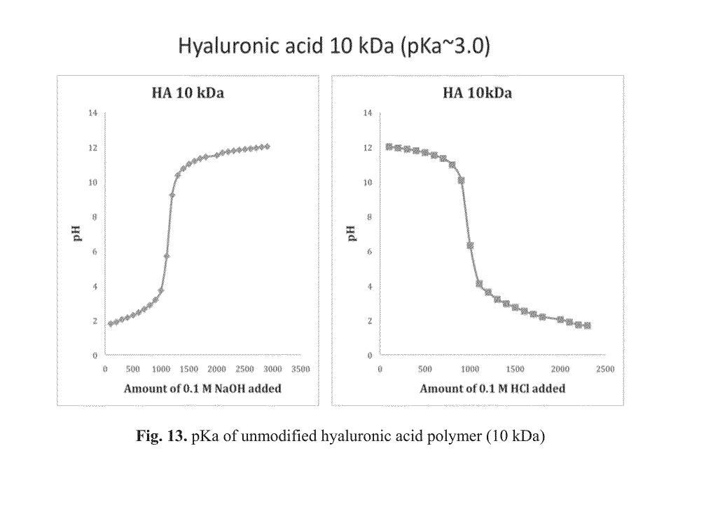

FIG. 13 provides acid-base titration curves of unmodified hyaluronic acid polymer (10 kDa) with a pKa of about 3.0.

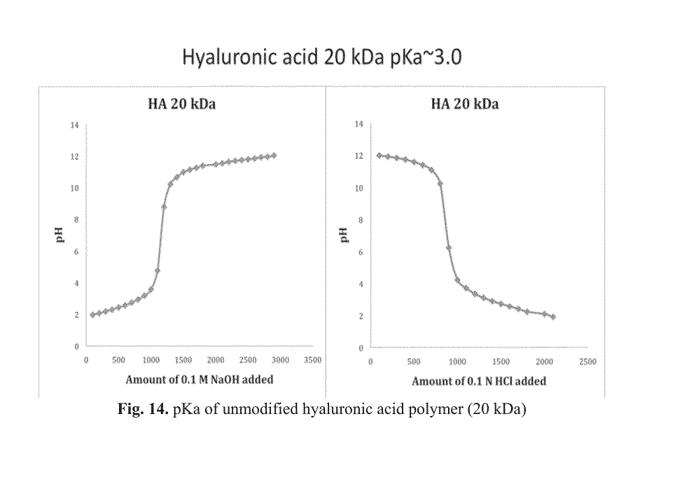

FIG. 14 provides acid-base titration curves of unmodified hyaluronic acid polymer (20 kDa) with a pKa of about 3.0.

FIG. 15 provides acid-base titration curves of hyaluronic acid polymer modified with oleyl amine (HA-OA) with a pKa of about 4.5.

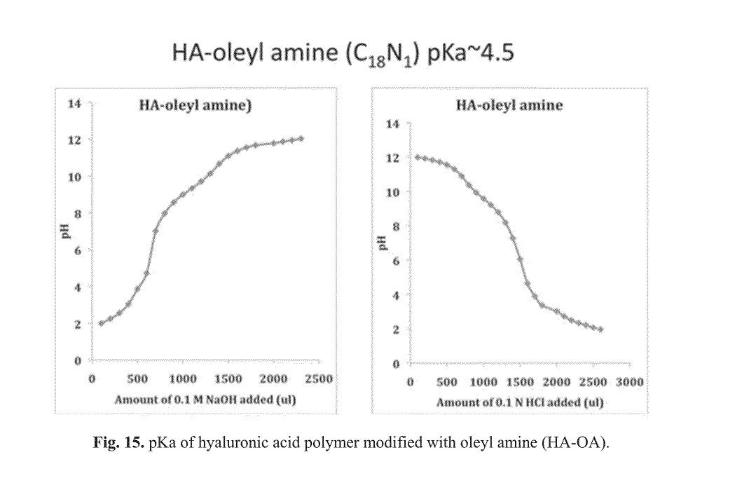

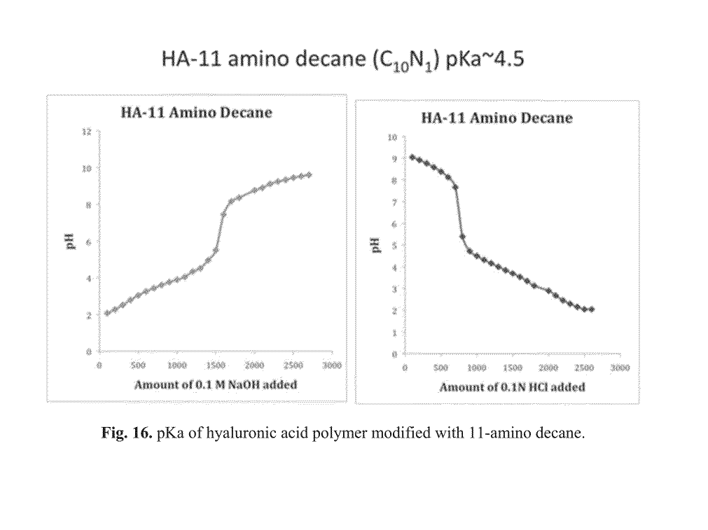

FIG. 16 provides acid-base titration curves of hyaluronic acid polymer modified with 1-amino decane with a pKa of about 4.5.

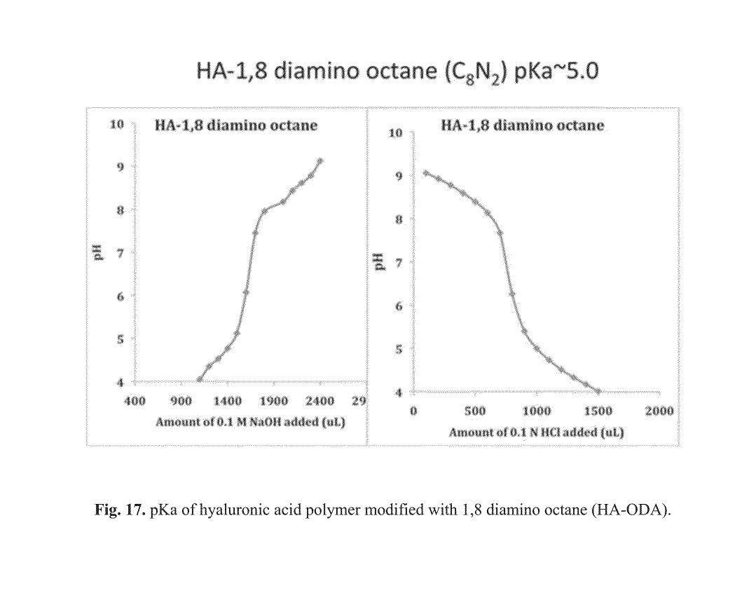

FIG. 17 provides acid-base titration curves of hyaluronic acid polymer modified with 1,8 diamino octane (HA-ODA) having a pKa of about 5.0.

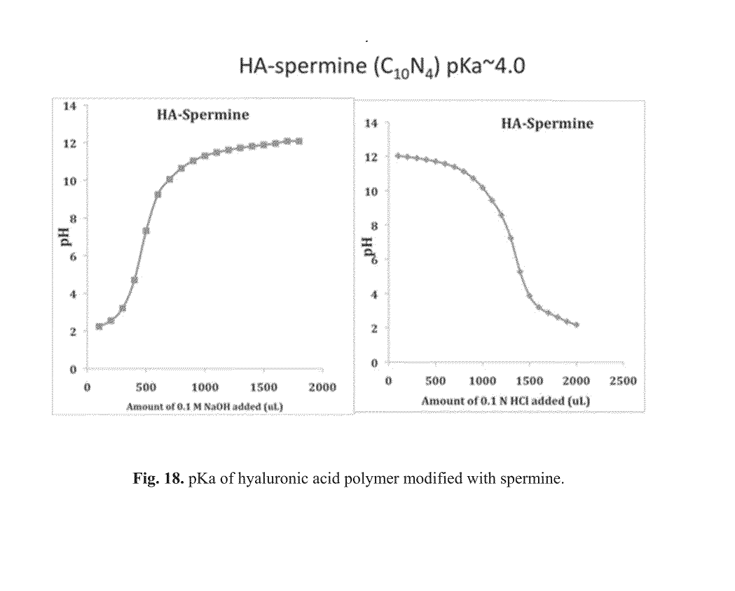

FIG. 18 provides acid-base titration curves of hyaluronic acid polymer modified with spermine with a pKa of about 4.

TABLE 1
pKa values of unmodified hyaluronic acid (HA) and some
of the HA-lipid modified polymer derivatives are shown.

| Hyaluronic acid and HA modified- | % Modification | |
|---|---|---|
| derivatives | (¹H-NMR) | pKa |
| HA 10 kDa | — | 3.0 |
| HA 20 kDa | — | 3.0 |
| HA₂₀-Oleyl amine (C₁₈N₁) | 10 | 4.5 |
| HA₂₀-Stearyl amine ((C₁₈N₁) | 15 | 4.7 |
| HA₁₀-Amino decanoic acid (C₁₀N₁) | 20 | 4.5 |
| HA₂₀-Amino decanoic acid (C₁₀N₁) | 20 | 4.5 |
| HA₁₀-Amino undecanoic acid (C₁₁N₁) | 20 | 4.5 |
| HA₂₀-Amino undecanoic acid (C₁₁N₁) | 20 | 4.5 |
| HA₁₀₋ 1,8 diamino octane (C₈N₂) | 20 | 5.0 |
| HA₂₀₋ 1,8 diamino octane (C₈N₂) | 15 | 4.8 |
| HA₁₀-(N,N-Dimethyldipropylene- | 20 | 4.8 |
| triamine)DMPA (C₆N₃) | | |
| HA₂₀-(N,N-Dimethyldipropylene- | 15 | 4.7 |
| triamine)DMPA (C₆N₃) | | |
| HA₂₀-Spermine (C₁₀N₄) | 08 | 4.0 |
| HA₁₀-Amino pipirazine (C₁₀N₄) | 20 | 5.0 |
| HA₂₀-Amino pipirazine (C₁₀N₄) | 10 | 4.5 |

7) Encapsulation of Drugs:

a) Encapsulation of Doxorubicin

PEGylated HA, alkyl HA and thiolated HA (5.3 mg each, prepared as described above) were dissolved in 1.5 ml of DI water and doxorubicin (4 mg, 20 weight percent) was added to this solution. The solution was homogenized at 6000 rpm for one minute. The product was dialyzed overnight against DI water with a MWCO of 14 kDa, centrifuged, and the supernatant was lyophilized.

b) Encapsulation of Taxol and Docetaxel

PEGylated HA, alkyl HA and thiolated HA were dissolved in water at a concentration of 1 mg/ml. The drug was dissolved in ethanol at 1 mg/ml. 4 ml of the drug solution was added to 16 ml of the HA derivative solution dropwise (20% by weight attempted drug loading). This cloudy mixture was allowed to stir for 24 hours at room temperature, ultrasonicated with a probe sonicator for three seconds every two seconds, and then centrifuged at 20,000×g for 10 minutes. The supernatant was collected, and ethanol was evaporated under vacuum. The product was lyophilized to yield nanoparticles in powder form.

c) Encapsulation of Etoposide:

PEGylated HA, alkyl HA and thiolated HA were dissolved in water at a concentration of 1 mg/ml. The drug was dissolved in methanol at 2 mg/ml. The drug solution was added to the HA derivative solution slowly, dropwise (20% by weight attempted drug loading). This mixture was allowed to stir for 24 hours at room temperature, ultrasonicated with a probe sonicator for three seconds every two seconds, and then centrifuged at 20,000×g for 10 minutes. The supernatant was collected, and methanol was evaporated under vacuum. The product was lyophilized to yield nanoparticles in powder form.

d) Encapsulation of Camptothecin:

PEGylated HA, alkyl HA and thiolated HA were dissolved in water at a concentration of 1 mg/ml. The drug was dissolved in DMSO at 2 mg/ml. The drug solution was added to the HA derivative solution slowly, dropwise (20% by weight attempted drug loading). This cloudy mixture was allowed to stir for 24 hours at room temperature, and then dialyzed for 24 hours. This product was ultrasonicated with a probe for 3 seconds every two seconds, and dialyzed against DI water for 24 hours. The solution was centrifuged at 20,000 g for 10 minutes and the supernatant was collected and lyophilized to yield a white powder.

e) Encapsulation of Topotecan:

PEGylated HA, alkyl HA and thiolated HA were dissolved in water at a concentration of 1 mg/ml. The drug was dissolved in water (1 mg/ml). The drug solution was added to the HA derivative solution slowly, dropwise (20% by weight attempted drug loading). This solution mixture was allowed to stir for 24 hours at room temperature, ultrasonicated with a probe sonicator for three seconds every two seconds, and then dialyzed for 24 hours. The product was lyophilized to yield nanoparticles in powder form.

f) Encapsulation of Idarubicin.

PEGylated HA, alkyl HA and thiolated HA were dissolved in water at a concentration of 1 mg/ml. The drug was dissolved in DMSO at 1 mg/ml. The drug solution was added to the HA derivative solution slowly, dropwise (20% by weight attempted drug loading). This cloudy mixture was allowed to stir for 24 hours at room temperature, ultrasonicated with a probe sonicator for three seconds every two seconds, and then dialyzed against DI water for 24 hours. This product was centrifuged at 20,000×g for 10 minutes and the supernatant was collected. The supernatant was collected, and the product was lyophilized to yield nanoparticles in powder form.

Transmission Electron Microscopic (TEM) Characterization of Drug Loaded HA Nanoparticles:

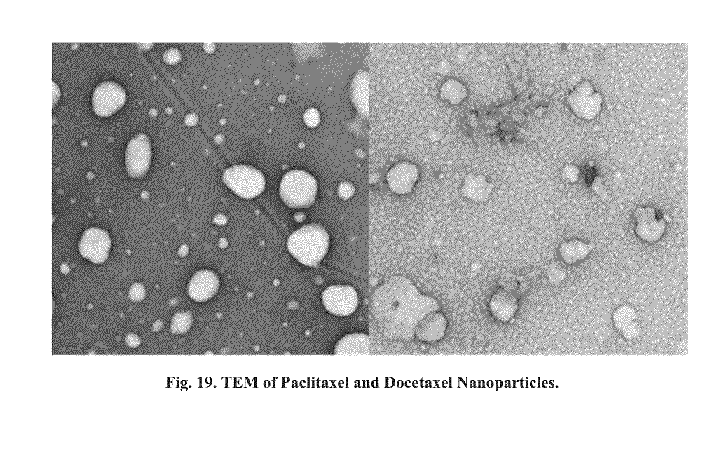

FIG. 19. TEM of Paclitaxel and Docetaxel Nanoparticles. The images show tetradecyl (C14) Paclitaxel-containing nanoparticles (left) and decyl (C10) Docetaxel-containing nanoparticles (right). The tetradecyl nanoparticles had a size of 227 nm, and the dodecyl nanoparticles had a diameter of 271 nm.

TABLE 2
Encapsulation of Diverse Drugs within Variable-Lipid Nanoparticles

| | | | Percent by | | | | | | Percent by | | |
|---|---|---|---|---|---|---|---|---|---|---|---|
| | | | weight | Encap- | Percent by | | | | weight | Encap- | Percent by |
| | Deriv- | Percent lipid | attempted | sulation | weight final | | Deriv- | Percent lipid | attempted | sulation | weight final |
| Drug | ative | modification | loading | efficiency | loading | Drug | ative | modification | loading | efficiency | loading |
| Doxorubicin | C4 | 20 | 20 | 100 | 20 | Docetaxel | C6 | 31.2 | 10 | 21 | 2.1 |
| | C6 | 20 | 20 | 100 | 20 | | C8 | 27.4 | 10 | 20 | 2 |
| logP | C8 | 20 | 20 | 100 | 20 | logP | C10 | 15.6 | 10 | 43 | 4.3 |
| −0.5 | C10 | 15.6 | 20 | 65 | 13 | 2.4 | C12 | 17.3 | 10 | 31 | 3.1 |
| | C12 | 17.3 | 20 | 72 | 14 | | C14 | 12.2 | 10 | 27 | 2.7 |
| | C14 | 12.2 | 20 | 67 | 13 | | C18 | 10.7 | 10 | 31 | 3.1 |
| | C18 | 10.7 | 20 | 65 | 13 | | C6 | 27.6 | 10 | 18 | 1.8 |
| Idarubicin | C6 | 31.2 | 10 | 100 | 10 | | C8 | 22.4 | 10 | 20 | 2 |
| | C8 | 27.4 | 10 | 23 | 2.3 | | C10 | 12 | 10 | 64 | 6.4 |
| logP | C10 | 15.6 | 10 | 55 | 5.5 | | C12 | 12 | 10 | 17 | 1.7 |
| 0.2 | C12 | 17.3 | 10 | 55 | 5.5 | | C14 | 12 | 10 | 3.2 | 0.32 |
| | C14 | 12.2 | 10 | 89 | 8.9 | | C18 | 12 | 10 | 30 | 3 |
| Topotecan | C6 | 31.2 | 10 | 71 | 7.1 | Paclitaxel | C6 | 31.2 | 10 | 30 | 3 |
| | C8 | 27.4 | 10 | 36 | 3.6 | | C8 | 27.4 | 10 | 19 | 1.9 |
| logP | C10 | 15.6 | 10 | 49 | 4.9 | logP | C10 | 15.6 | 10 | 28 | 2.8 |
| 0.8 | C12 | 17.3 | 10 | 98 | 9.8 | 3 | C12 | 17.3 | 10 | 32 | 3.2 |
| | C14 | 12.2 | 10 | 49 | 4.9 | | C14 | 12.2 | 10 | 18 | 1.8 |
| | C18 | 10.7 | 10 | 59 | 5.9 | | C18 | 10.7 | 10 | 11 | 1.1 |
| Etoposide | C6 | 31.2 | 20 | 62 | 12 | | C6 | 27.6 | 10 | 20 | 2 |
| | C8 | 27.4 | 20 | 40 | 8 | | C8 | 22.4 | 10 | 23 | 2.3 |
| logP | C10 | 15.6 | 20 | 67 | 13 | | C10 | 12 | 10 | 7.2 | 0.72 |
| 1 | C12 | 17.3 | 20 | 66 | 13 | | C12 | 12 | 10 | 18 | 1.8 |
| | C14 | 12.2 | 20 | 57 | 11 | | C14 | 12 | 10 | 3.5 | 0.35 |
| | C18 | 10.7 | 20 | 61 | 12 | | C18 | 12 | 10 | 32 | 3.2 |
| Camptothecin | C6 | 31.2 | 10 | 32 | 3.2 | | | | | | |
| | C8 | 27.4 | 10 | 50 | 5 | | | | | | |
| logP | C10 | 15.6 | 10 | 23 | 2.3 | | | | | | |
| 1.74 | C12 | 17.3 | 10 | 12 | 1.2 | | | | | | |
| | C14 | 12.2 | 10 | 21 | 2.1 | | | | | | |
| | C18 | 10.7 | 10 | 16 | 1.6 | | | | | | |

Encapsulation of Diverse Drugs within Variable-Lipid Nanoparticles.

Table 2 shows the encapsulation of drugs in different HA derivatives with varying lipid modification. Attempted weight by loading and encapsulation efficiency resulted in percent by weight final loading.

8) In Vitro Cell Up Take Studies of Free Doxorubicin (DOX) and DOX Loaded HA Polymers:

In order to determine the pH altering effects of the hyaluronic acid-lipid modified polymers and cellular trafficking of drugs, a model fluorescent drug, doxorubicin, was encapsulated into the unmodified and modified HA polymers and tested on MDA-MB-231 breast cancer cell lines.

Method:

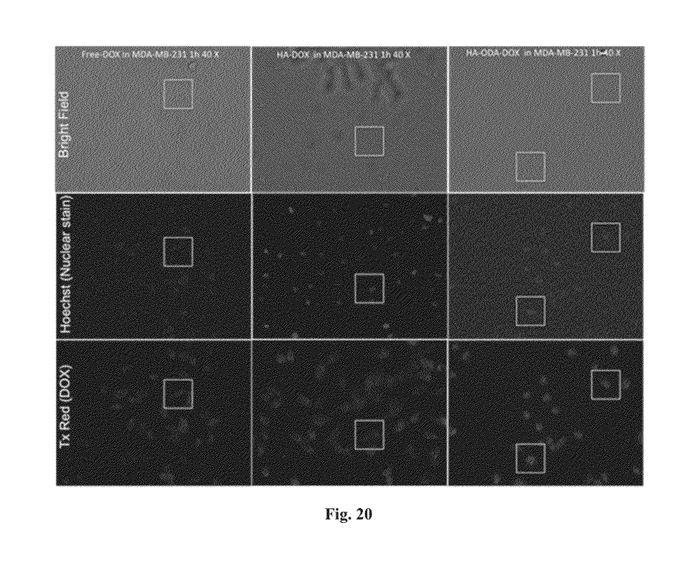

The MDA-MB-231 cells were grown in 6 well culture plates and incubated with equimolar concentration (20 μM) of either the free doxorubicin (DOX), DOX encapsulated in the unmodified hyaluronic acid polymer (HA, 10 kDa, pKa˜3) or DOX encapsulated in one of the lipid-modified HA derivative, HA-1,8 diamino octane (HA-ODA, pKa˜5). After 1 h of incubation, the cells were washed thrice with PBS and the uptake of DOX in the cells was observed using a fluorescence microscope. The results are shown in FIG. 20, which shows fluorescence microscopy of free doxorubicin (DOX), DOX loaded unmodified hyaluronic acid (HA-DOX, PKa˜3) polymer and DOX loaded HA polymer modified with 1,8 diamino octane (HA-ODA-DOX, PKa˜5); magnification used, 40×.

From the figure it can be seen that after 1 h incubation of free DOX or DOX loaded unmodified and lipid modified HA derivative, there was significantly higher uptake of DOX in the nucleus of the cells treated with HA-ODA-DOX formulation, however the uptake of DOX in the cells treated with either free DOX or unmodified HA-DOX formulation were not significantly different, wherein the uptake of DOX in the cells is found to be higher in the cytoplasm and endosomal/lysosomal compartments and much lower in the nucleus. This effect may be in part due to the efficient uptake of the drug within 1 h into the cells by both the unmodified polymer and the lipid-modified derivative but the higher pKa value of the HA-ODA derivative may have facilitated the endosomal/lysosomal escape of the encapsulated DOX and its efficient translocation into the nucleus.

9) Cytotoxicity Evaluation of Drug Loaded HA Nanoparticles on Ovarian Cancer Cells:

TABLE 3
IC₅₀ of Nanoparticles vs. IC₅₀ of Free Drug. The IC₅₀ of
the encapsulated drug and the free drug were determined by
cytotoxicity assays using SKOV3 cells at a 48 hour timepoint.

| | Free drug | Encapsulated drug |
|---|---|---|
| Drug | IC₅₀ (μM) | IC₅₀ (μM) |
| Doxorubicin | 5.784 | 1.094 |
| Idarubicin | 3.82 | 4.2 |
| Topotecan | 0.22 | 0.73 |
| Etoposide | 4.58 | 3.37 |
| Camptothecin | 0.82 | 0.29 |
| Paclitaxel | 0.0101 | 0.00789 |

The general trend seen was that IC₅₀ is improved in the case of the encapsulated drug.

10) Encapsulation of Oligonucleotides/siRNA in HA Based Nanoparticles:

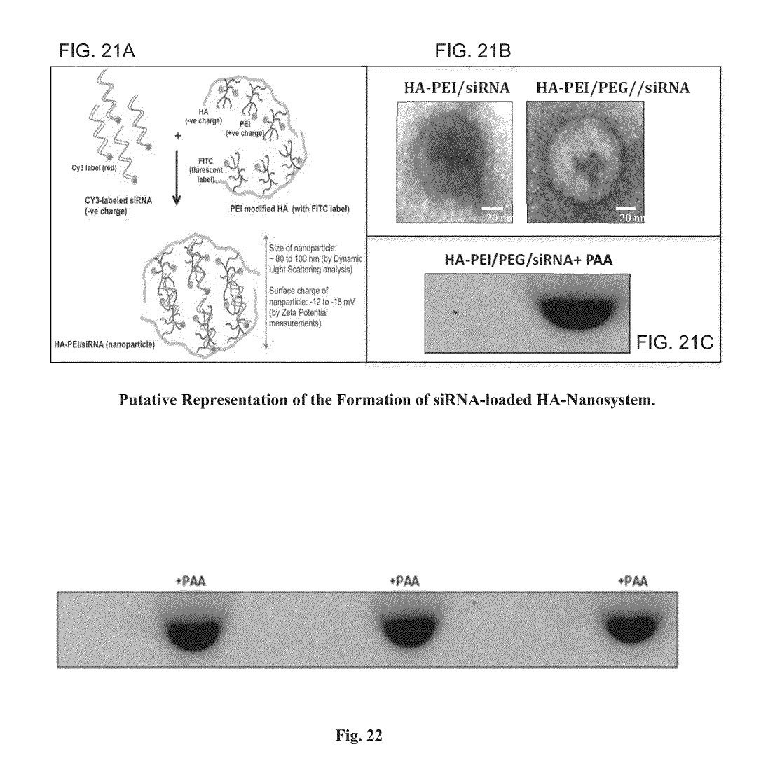

FIGS. 21A-C provide a putative representation of the formation of siRNA-loaded HA-nanosystem. The negatively charged siRNA is proposed to complex with the cationic polymer PEI forming the core, with the negatively charged HA forming the corona (A). The proposed structure is supported by the size and charge data from light scattering and transmission electron microscopy (B). Also 100% siRNA loading in HA nanosystem was observed by gel retardation assay that could be released in presence of polyacrylic acid (PAA).

Determination of siRNA Entrapment Efficiency and Release Using Gel Retardation Assays

To confirm if the particles encapsulated siRNA, an agarose gel electrophoresis was utilized (FIG. 22). The polymer/siRNA complex was prepared by mixing HA derivative with siRNA and incubating at RT for 30 min. These complexes were run on gel and determined the mean density of siRNA bands. More specifically, FIG. 22 provides electrophoretic retardation analysis of siRNA binding by HA-PEI derivatives at different mass ratios (90:1, 54:1, 45:1). The release of intact siRNA by polyacrylic acid was shown in each case.

The binding percentage was calculated based on the relative intensity of free siRNA band in each well with respect to wells with free siRNA (in the absence of any polymers). When there was complete complexation, the free band completely disappeared. In cases when there was complete complexation and there was no free band on gel, an alternate method was utilized to confirm that there was siRNA encapsulation. Complexes were treated with a polyanionic poly acrylic acid and run on gel. This anionic poly(acrylic acid) (PAA) would compete with the anionic polymer and release the siRNA which then appears as a free band in the gel. The ability of complexes to release siRNA after a challenge with the competing polyanionic PAA was determined by measuring the mean density of siRNA band that appear after the treatment. When particles were treated with poly acrylic acid, complexes with and without PAA were run on gel to confirm that the siRNA was intact when it was complexed.

11) In Vitro siRNA Delivery

TABLE 4
The siRNA delivery efficiency into tumor cells was tested
using various HA-lipid formulations as below:

| | Self | siRNA | Activ- | Size | Charge |
|---|---|---|---|---|---|
| | assem- | encap- | ity in | (nm) + | (mV) + |
| HA Derivative | bly | sulation | cells | siRNA | siRNA |
| HA- butylamine | − | − | − | — | — |
| in water (C4) | | | | | |
| HA- hexylamine | + | − | − | 1000 ± 1 | −20 |
| in water (C6) | | | | | |
| HA- octylamine | + | − | − | 200 ± 0.3 | −20 |
| in water (C8) | | | | | |
| HA-stearylamine | + | − | − | 190 ± 0.3 | −15 |
| in water (C18) | | | | | |
| HA- 1,6 | + | + | − | 320 ± 0.5 | −8 |
| diaminohexane | | | | | |
| in water | | | | | |
| HA-1,8 diaminooc- | + | + | − | 142 ± 0.2 | −10 |
| tane in water | | | | | |
| HA- choline in | + | + | − | 175 ± 0.4 | 0 |
| water | | | | | |
| HA-spermine in | + | + | + | 190 ± 0.3 | +16.5 |
| water | | | | | |
| HA-polyeth- | + | + | + | 50 ± 0.9 | −6.5 |
| yleneimine | | | | | |
| (PEI) in PBS | | | | | |
| HA-PEI/HA-PEG | + | + | + | 85 ± 0.9 | −5.5 |
| in PBS | | | | | |
| HA-PEI/HA-PEG/ | + | + | + | 90 ± 1.2 | −8.5 |
| HA-SH in PBS | | | | | |

11) Preliminary Evaluations of Delivery in Cells Expressing CD44 Receptors

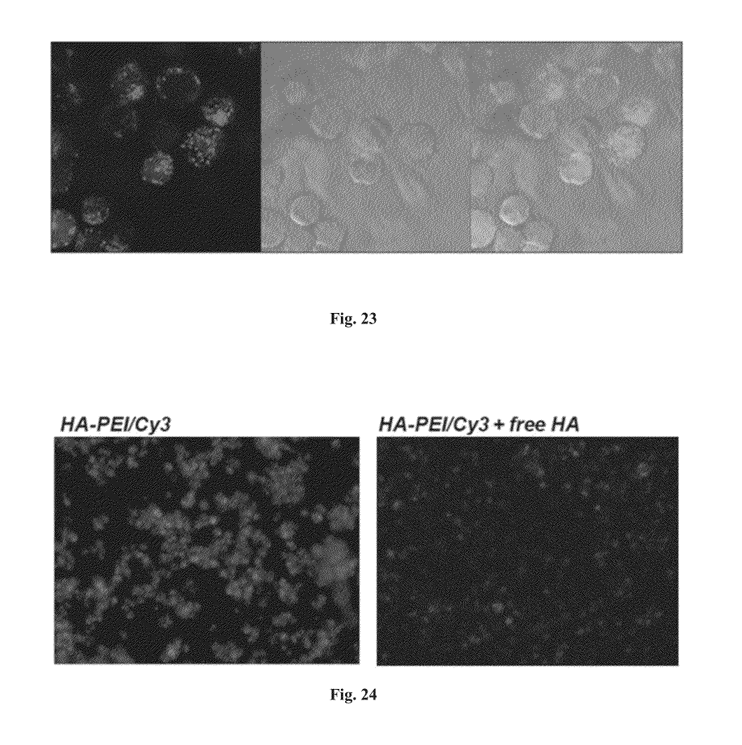

The derivatives that formed good size nanoparticles and demonstrated good siRNA encapsulation were taken forward to evaluate the activity in cells. The prepared Cy3siRNA/polymer complexes were reverse transfected into cells expressing CD44 (MDA MB-468) at 50 nM siRNA concentration and incubated for 48 hours and examined under the confocal microscope to see if there was any cell uptake. FIG. 23 provides the confocal microscopy images of MDAMB-468 cells after treatment with HA PEI/Cy3siRNA at 50 nM for 12 h. The internalization of siRNA could be clearly seen in the cells (red signal).

For competitive inhibition studies to determine the cellular uptake of HA nanoparticles, the cells were pre-treated with 2 ml of serum free culture medium containing HA at 10 mg/ml. After the treatment, the Cy3 labeled HA nanoparticles were added to the MDA-MB 468 cells followed by incubation of 24 hours. The cells were washed twice with PBS and examined under microscope. A large reduction in cell uptake was noticed in the cells that were pre-treated with excess HA, suggesting that these particles enter into cells by receptor mediated pathway. No activity was detected in cells that do not express CD44, again confirming that this is a receptor mediated pathway. FIG. 24 shows the results of the competitive inhibition study, where the cells were incubated with HA-PEI/Cy3-labeled siRNA in the presence and absence of excess free HA.

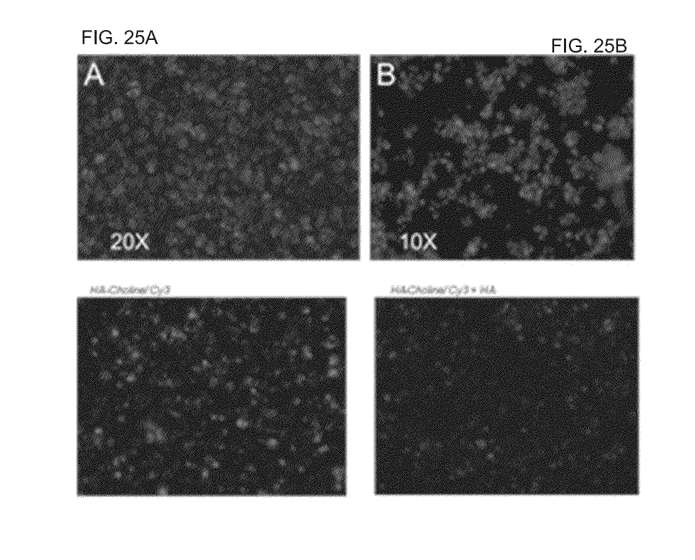

A similar result was observed with HA-choline derivatives. FIGS. 25A-B show cellular uptake of HA-choline/cy3 siRNA in MDA-MB 468 cells. For competitive inhibition study, the cells were incubated with HA-choline in the presence and absence of excess free HA

12) Effects of Chloroquine in Enhancing Endosomal Escape

Cell uptake studies showed that the hydrophobically modified derivatives of HA, despite their resultant negative charge, entered into cells but gave no cellular activity. It has been demonstrated previously that the cell entry was receptor mediated and it is independent of the charge on the surface. The presence of positive charge was most likely to help the complex to get out of the endosome. All the derivatives described herein, except the HA-SP and HA-PEI, demonstrated cell uptake but no gene down regulation.

In order to confirm that these complexes are stuck in the endosome without being released, the transfection was done in the presence of a weak base, chloroquine. It has been reported that this small molecule helps to disrupt the endosome in addition to inhibit the endosome-lysosome fusion. Both of these activities together appear to help the complex to be released from the endosome. Treatment of cells with HA-SP/siRNA (at 90:1 ratio) and chloroquine demonstrated activity in cells whereas the same complex without chloroquine failed to show cell activity.

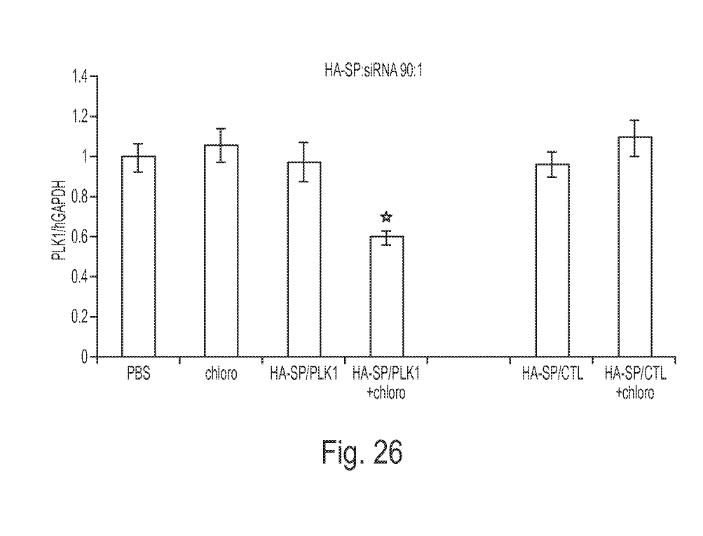

The results are provided in FIG. 26, which shows HA-SP/PLK1 siRNA mediated PLK1 gene silencing in the presence of chloroquine in MDA MB 468 cells at 90:1 ratio. Cells treated with PLK1 siRNA formulated HA-SP or CTL siRNA formulated HA-SP in the presence or absence of chloroquine for 48 hours. The PLK1 gene expression was measured by qPCR. Data represented as a mean±SD (n=3). *p=0.01 compared to PBS and CTL treatment groups.

13) In Vitro Gene Silencing Studies

After confirming the cell uptake, the ability of this HA-spermine complex to deliver a functional siRNA was evaluated using PLK1 targeted siRNA to inhibit PLK1 gene expression in CD44 expressing cells. Cells were transfected with different HA derivative/siRNA and at different concentrations (50-300 nM). After 48 hours, the RNA was extracted from the cells and subjected to quantitative PCR to determine the mRNA knockdown. Although all the fatty acid modified HAs have demonstrated cell uptake, most of them failed to down regulate the PLK1 gene expression.

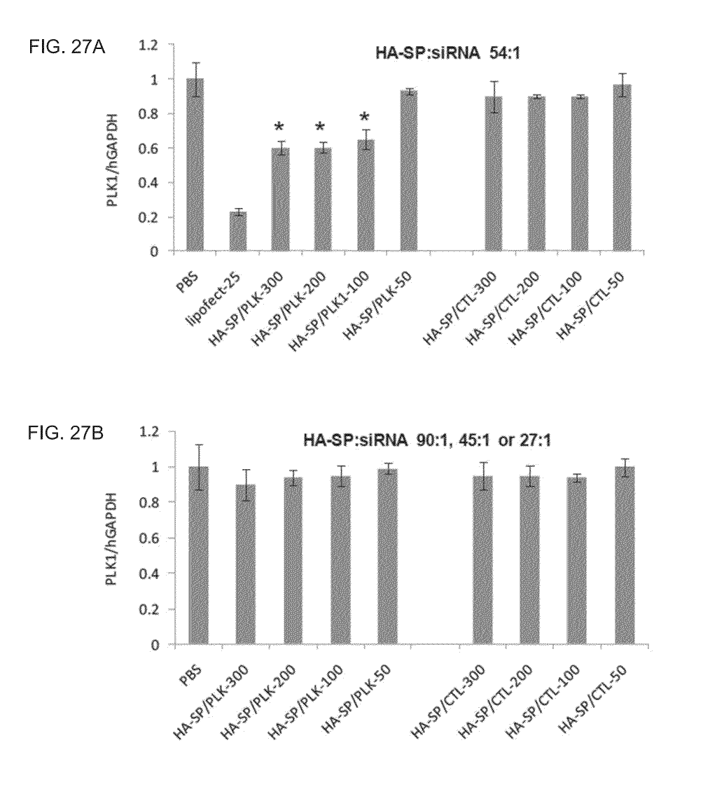

The spermine derivatized HA demonstrated about 40% activity at 100, 200 and 300 nM while the control siRNA/HA-SP in the same study did not produce any activity (FIGS. 27A-B). It's interesting to note that the HA-SP demonstrated activity only at the mass ratio of 54:1 (polymer:siRNA). It failed to demonstrate activity at a ratio of 18:1 (polymer:siRNA) or higher. It's also worth noting that the zeta potential of the 55:1 ratio complex was around +16.5 mV whereas the other one was around +5-6 mV or close to neutral.

More specifically, FIG. 27 shows HA-SP/PLK1 siRNA mediated PLK1 gene silencing in MDAMB468 cells. Cells treated with PLK1 siRNA formulated HA-SP or CTL siRNA formulated HA-SP for 48 hours at mass ratios (1)54:1(A) or (2) 45: or 27:1. (B). The PLK1 gene expression was measured by qPCR. Data represented as mean±SD(n=3). *p=0.01 compared to PBS and CTL treatment groups.

Since it's believed that these complexes enter into the cells by receptor mediated pathways, the resultant positive charge on the surface probably helps the complex to get out of the endosome. In addition to HA-SP, the PEI modified HA also demonstrated activity in the CD44 expressing MDA-MB 468 cells. Again, at the ratio of 54:1, the complex demonstrated good activity with good dose response. This complex also failed to show activity at 27:1, 18:1 or 9:1 ratios.

In contrast to the HA-SP/siRNA complex, the HA-PEI became completely negative in charge after encapsulating the siRNA. Nonetheless, the complex showed good activity in cells, which suggests the core/shell structure of the HA-PEI/siRNA complex with HA backbone exposed in the shell and the PEI grafted chains complexed with siRNA molecules in the core.

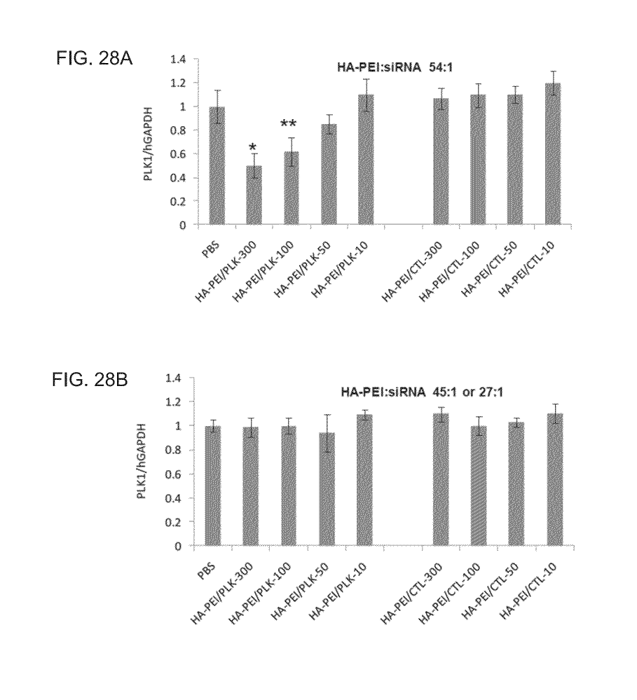

FIGS. 28A-B show the results for HA-PEI/PLK1 siRNA mediated PLK1 gene silencing in MDAMB468 cells. Cells treated with PLK1 siRNA formulated HA-PEI or CTL siRNA formulated HA-PEI for 48 hours at mass ratios (1) 54:1 or (2) 45:1 or 27:1. The PLK1 gene expression was measured by qPCR. Data represented as mean±SD(n=3). *p=0.01 and **p=0.02 compared to PBS and CTL treatment groups.

14) PLK1 siRNA Knockdown in A549/A549^{DDP} NSCL Cancer Cells

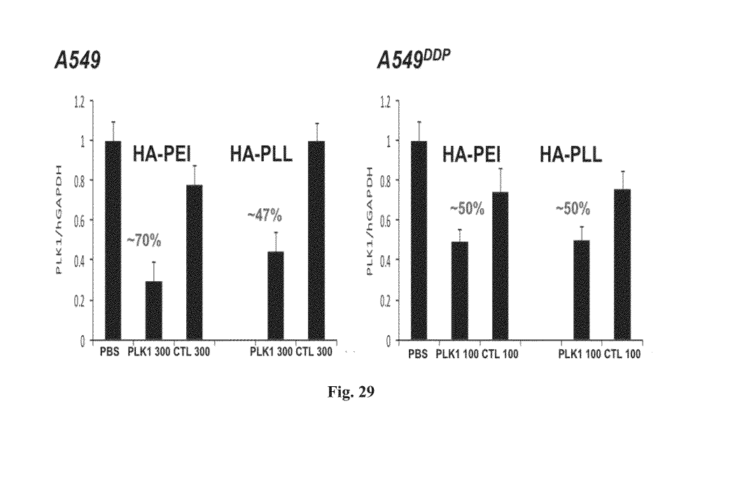

Non-small cell lung cancer cells were transfected with PLK1 siRNA encapsulated in HA-PEI or HA-PLL nanosystems at a concentration of 100 and 300 nM. Cells were harvested and RNA was extracted after 48 hrs. qPCR was run to determine the target gene knockdown. As seen in FIG. 29, HA-PEI and HA-PLL both could stably encapsulate PLK1 siRNA and exhibit marked downregulation of target gene in both sensitive and resistant lung cancer cells.

15) Live Animal Imaging and Tumor Targeting of HA Based Nanosystems.

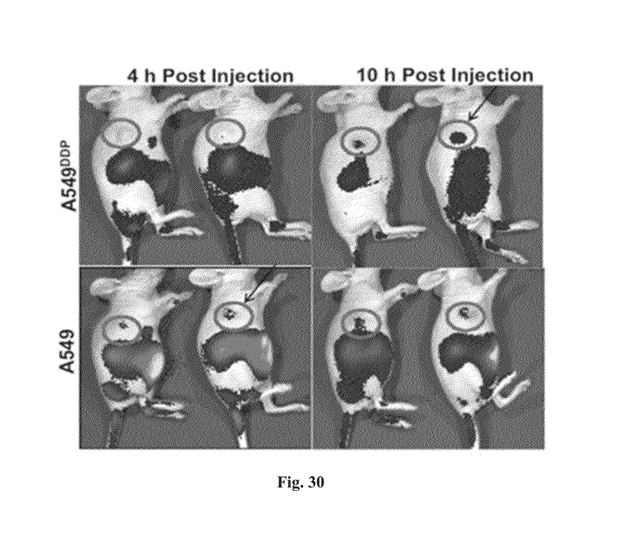

CD44 expressing A549 and A549^{DDP} sensitive and resistant NSCL cancer bearing mice were imaged with indocyanine green (ICG) dye loaded HA nanosystem at the time shown. Time dependent accumulation of the dye loaded HA nanosystems in the tumor is clearly seen in FIG. 30.

16) Tissue Distribution of Survivin siRNA in A549^{DDP} Tumor Bearing Mice.

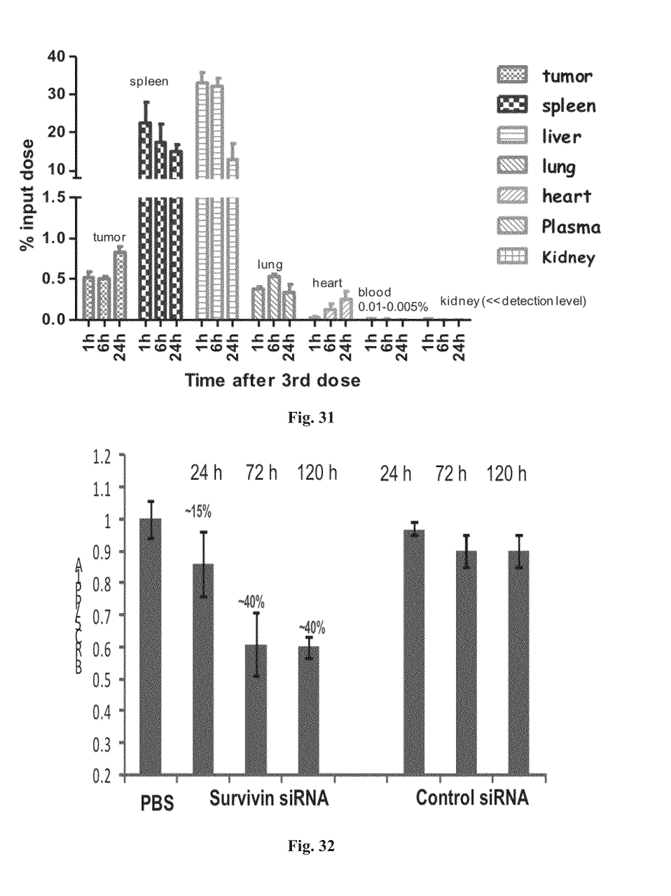

Mice were injected thrice with HA-PEI/PEG/survivin at a dose of 0.5 mg/kg and subsequently the tissues were collected at 1, 6 and 24 hours after the last dose. PCR method was utilized to quantitate the siRNA in tissue samples. Results are provided in FIG. 31.

17) In Vivo Gene Silencing: Survivin Gene Knockdown in A549^{DDP} Tumors at Varying Time Points.

Tumor bearing mice were intravenously injected with survivin siRNA encapsulated in HA-PEI/HA-PEG nanoparticles at a dose of 0.5 mg/kg for 3 days. At 24, 72 and 120 hours after injection, tumors were harvested and RNA was extracted. qPCR was run to determine the target KD. As shown in FIG. 32, the target gene knock down up to 5 days was observed.

18) Combination siRNA/Drug Efficacy and Safety

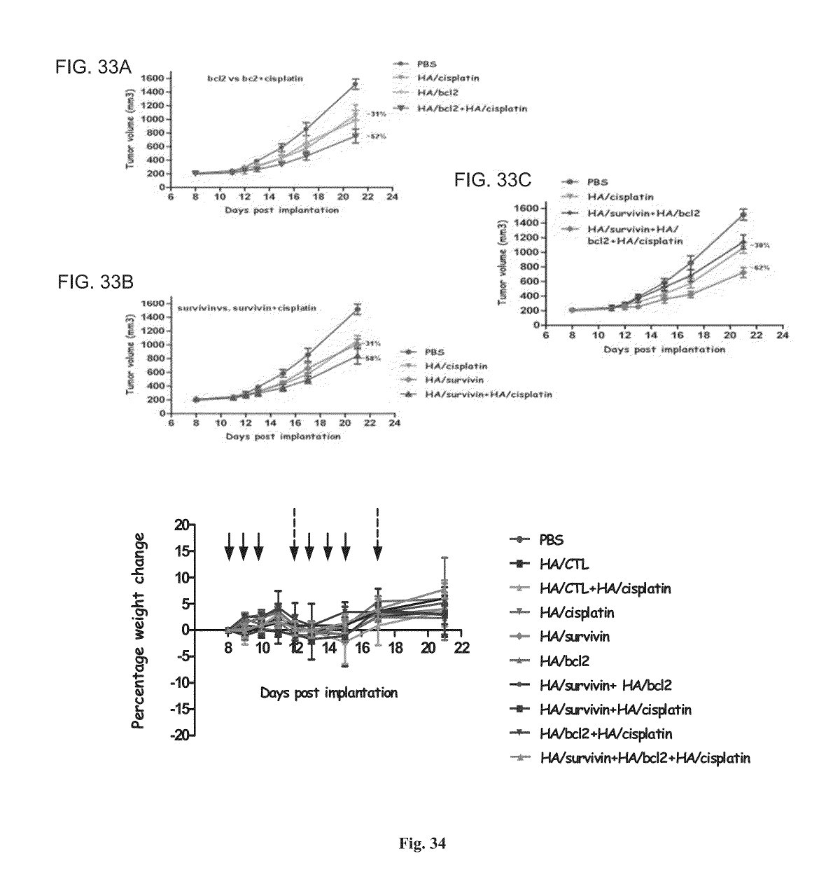

FIGS. 33A-C provide results relating to efficacy of a combination siRNA and Cisplatin loaded in HA Nanosystems. A) HA-ODA/Cisplatin or its combination with bcl2 siRNA in HA-PEI nanosystem is shown; B) HA-ODA/Cisplatin or its combination with survivin siRNA in HA-PEI Nanosystem is shown; and C) The effect of HA-ODA/Cisplatin nanosystem or its combination with both bcl2 and survivin loaded in HA-PEI nanosystem is shown. From the results, it is clear that the combination treatment exhibits the best synergism in terms of tumor suppression.

In Vivo Safety Evaluation of HA Nanosystems.

FIG. 34 shows the % body weight change in mice that had single or combination treatment during the study period. It is evident from the results that at the administered dose of HA nanoparticles, no apparent toxicity or weight loss was observed indicating the safety of the delivery systems in vivo.

What is claimed is:

1. A composition comprising a water-soluble polymeric buffer, wherein the polymeric buffer comprises a biodegradable pH altering polymer or a biodegradable polymer conjugated with pH altering groups.

2. The composition of claim 1, wherein the pH altering groups comprise at least one nitrogen-containing group.

3. The composition of claim 2, wherein the pH altering groups comprise at least one amine group.

4. The composition of claim 1, wherein at least one of the pH altering groups is derivative of a compound selected from the group consisting of butyl amine, hexyl amine, octyl amine, stearyl amine, 1,3 diamine propane, 1,4 diamino butane, 1,6 diamino hexane, 1,2 aminoethyl piperazine and spermine.

5. The composition of claim 1, wherein the biodegradable polymer is selected from the group consisting of hyaluronic acid (HA) and dextran.

6. The composition of claim 1, wherein the polymeric buffer is selected from the group consisting of HA-butylamine, HA-hexylamine, HA-octylamine, HA-1-amino decane, HA-stearylamine, HA-oleyl amine, HA-1,6 diaminohexane, HA-1,8 diaminooctane, HA-choline, HA-polyethyleneimine and HA-spermine.

7. The composition of claim 1 wherein the polymeric buffer comprises primary, secondary or tertiary nitrogen containing molecules.

8. The composition of claim 6 further comprising a therapeutic agent.

9. The composition of claim 7 wherein the therapeutic agent is encapsulated in the polymeric buffer.

10. A pharmaceutical composition comprising a pharmaceutically-acceptable carrier or diluent and at least one polymeric buffer, wherein the polymeric buffer comprises a biodegradable pH altering polymer or a biodegradable polymer conjugated with pH altering groups.

11. The pharmaceutical composition of claim 10 further comprising a therapeutic agent.

12. The pharmaceutical composition of claim 11, wherein the therapeutic agent is a chemotherapeutic agent.

13. The pharmaceutical composition of claim 10, wherein the chemotherapeutic agent is doxorubicin.

14. The pharmaceutical composition of claim 10, wherein the biodegradable polymer is water soluble.

15. The pharmaceutical composition of claim 10, wherein the biodegradable polymer is selected from the group consisting of hyaluronic acid (HA) and dextran.

16. A method of treating a subject having a tumor, the method comprising administering to the subject a composition containing nanoparticles in an amount sufficient to reduce tumor size or number of tumor cells in the tumor, wherein the nanoparticle comprises:
a) a therapeutic agent; and
b) a hydrogel shell surrounding the therapeutic agent, the hydrogel shell comprising biodegradable pH altering polymer or a biodegradable polymer conjugated with pH altering groups,
thereby treating the subject.

17. A method of inhibiting expression of a target polypeptide in a subject, the method comprising administering to the subject a composition containing nanoparticles in an amount sufficient to inhibit expression of the target polypeptide, wherein the nanoparticle comprises:
a) a core comprising a functional nucleic acid or functional nucleic acid construct; and
b) a hydrogel shell surrounding the core, the hydrogel shell comprising a biodegradable pH altering polymer or a biodegradable polymer conjugated with pH altering groups, thereby inhibiting the expression of the target polypeptide.

18. The method of claim 17 wherein the core comprises siRNA.

19. A method of modifying pH of a microenvironment in need of pH modification comprising introducing into the microenvironment a polymeric buffer, wherein the polymeric buffer comprises a biodegradable pH altering polymer or a biodegradable polymer conjugated with pH altering groups.

20. The method of claim 19, wherein the pH altering groups comprise at least one nitrogen-containing group.

21. The method of claim 20, wherein the pH altering groups comprise at least one amine group.

22. The method of claim 19, wherein at least one of the pH altering groups is derivative of a compound selected from the group consisting of butyl amine, hexyl amine, octyl amine, stearyl amine, 1,3 diamine propane, 1,4 diamino butane, 1,6 diamino hexane, 1,2 aminoethyl piperazine and spermine.

23. The method of claim 19, wherein the biodegradable polymer is selected from the group consisting of hyaluronic acid (HA) and dextran.

24. The method of claim 19, wherein the polymeric buffer is selected from the group consisting of HA-butylamine, HA-hexylamine, HA-octylamine, HA-1-amino decane, HA-stearylamine, HA-oleyl amine, HA-1,6 diaminohexane, HA-1,8 diaminooctane, HA-choline, HA-polyethyleneimine and HA-spermine.

25. The method of claim 19 further comprising introducing a therapeutic agent to the microenvironment.

26. The method of claim 25 wherein the therapeutic agent is encapsulated in the polymeric buffer.
